(19) United States
(12) Patent Application Publication
(10) Pub. No.: US 2026/0251614 A1
(43) Pub. Date: Aug. 27, 2026

(54) METHOD AND SYSTEM FOR REAL-TIME DETECTION AND QUANTIFICATION OF PER- AND POLYFLUOROALKYL SUBSTANCES

(71) Applicant: Texas Tech University System, Lubbock, TX (US)

(72) Inventors: Gerardine G. BOTTE, Lubbock, TX (US); Dayana Donneys VICTORIA, Lubbock, TX (US); Juan Carlos MUNOZ SENMACHE, Lubbock, TX (US)

(73) Assignee: TEXAS TECH UNIVERSITY SYSTEM, Lubbock, TX (US)

(21) Appl. No.: 19/549,622

(22) Filed: Feb. 25, 2026

Related U.S. Application Data
(60) Provisional application No. 63/762,921, filed on Feb. 25, 2025.

(51) Int. Cl. G01N 27/48; G01N 27/30; G01N 27/416

(52) U.S. Cl. CPC G01N 27/48; G01N 27/308; G01N 27/4161

(57) ABSTRACT

A system and method for real-time electrochemical detection of per- and polyfluoroalkyl substances (PFAS). The system includes an electrochemical sensor having a working electrode, a counter electrode, and a reference electrode, where the working electrode comprises a transition metal or a graphene-metal composite. The system includes a controller configured to regulate an applied voltage and measure electrochemical response. The system includes a fluid handling system configured to transport a sample containing PFAS to the electrochemical sensor. The system includes a data processing system configured to analyze the electrochemical response and determine PFAS concentration based on a calibration model. The test kit system and method can be utilized for electrochemical detection of PFAS in aqueous samples.

### CROSS-REFERENCE TO RELATED PATENT APPLICATIONS

The application claims priority to U.S. Patent Appl. Ser. No. 63/762,921, to Gerardine G. Botte, et al., entitled “Method and System for Real-Time Detection and Quantification of Per- and Polyfluoroalkyl Substances Using an Ultra-Fast Electrochemical Sensor,” filed Feb. 25, 2025, which patent application is commonly owned by the owner of the present invention and is incorporated herein in its entirety.

### STATEMENT OF FEDERALLY FUNDED RESEARCH

This disclosure is related to federally sponsored research and development under the government funding from NSF 20-553 Gen-4 Engineering Research Centers Grant Award No. 2133576.

The invention was made with United States government support. The United States government has certain rights in the invention.

### TECHNICAL FIELD

The present disclosure relates to the field of electrochemical sensing and environmental monitoring. In particular, the present disclosure relates to an ultra-fast electrochemical sensor for real-time detection and quantification of per- and polyfluoroalkyl substances (PFAS) in water and solid samples.

The present invention also relates to test kit systems and methods for the electrochemical detection of PFAS, and more particularly, to pre-packaged reagent kits for use with graphene-coated transition metal electrodes for field-deployable measurement of PFOA and PFOS in drinking water at concentrations at or below 4 parts per trillion (ppt).

### BACKGROUND

The presence of per- and polyfluoroalkyl substances (PFAS) in water supplies and biosolids poses a significant risk to human health and the environment. PFAS are persistent, bioaccumulative, and toxic, making their detection and removal a critical priority for regulatory agencies and environmental monitoring efforts.

The United States Environmental Protection Agency (EPA) established maximum contaminant levels (MCLs) for six PFAS compounds, setting stringent limits between 4 and 10 parts per trillion (ppt). These regulatory standards necessitate the development of accurate and field-deployable detection technologies capable of ultra-trace quantification of PFAS in complex environmental matrices.

Conventional PFAS detection relies primarily on high-performance liquid chromatography coupled with tandem mass spectrometry (HPLC-MS/MS) and ultra-performance liquid chromatography-quadrupole time-of-flight high-resolution mass spectrometry (UPLC-QTOF-HRMS).

While these techniques achieve the necessary detection limits, they require costly instrumentation, specialized expertise, and extensive sample preparation. Additionally, centralized laboratory analysis results in long turnaround times, preventing real-time monitoring in high-risk areas such as industrial discharge sites, wastewater treatment facilities, and agricultural irrigation networks. The complexity and expense of these methods create significant barriers to widespread PFAS monitoring and regulatory compliance.

There is a need for detection strategies that can provide low cost, rapid response times, and portability. Molecularly imprinted polymer (MIP)-based electrochemical sensors have demonstrated detection sensitivities in the range of 20 ppt, but they suffer from slow kinetics, electron mediator dependencies, and inconsistent performance in real-world environmental samples. Other electrochemical platforms rely on surface-functionalized electrodes but face challenges in distinguishing between structurally similar PFAS compounds. Existing graphene-based electrochemical sensors have been explored for PFAS detection, but they typically operate at detection limits exceeding 4000 ppt, far above regulatory requirements. Additionally, their broad operating potential windows introduce potential interference from competing electrochemical reactions, limiting their reliability.

The current technological landscape lacks a rapid, field-deployable electrochemical sensor capable of detecting and quantifying PFAS at EPA-mandated concentrations without requiring extensive sample preparation or specialized reagents.

Therefore, there is a need to combine ultra-trace detection capabilities with simplified operational requirements, enabling real-time PFAS monitoring across diverse environmental conditions.

Furthermore, as current U.S. regulations for PFAS in drinking water establish maximum contaminant levels of 4 ppt for perfluorooctanoic acid (PFOA) and 4 ppt for perfluorooctanesulfonic acid (PFOS), the detection at these ultra-trace concentrations requires analytical methods with high sensitivity and reproducibility. Existing methods such as LC-MS/MS require expensive laboratory equipment, trained personnel, and sample shipping, resulting in turnaround times of days to weeks.

Electrochemical sensors based on graphene-coated nickel electrodes, as described in EP 3 060 701 B1 (incorporated herein by reference), provide a platform for the catalytic detection of analytes through the formation of nickel oxyhydroxide (NiOOH) and other transition metals in alkaline media.

For field deployment, the reagent solutions are pre-packaged, volume-matched, and concentration-matched to ensure that the only variable between blank and sample measurements is the presence or absence of PFAS.

Accordingly, there is a need for test kit methods and systems that provides all necessary reagent solutions in pre-measured volumes with matched dilution factors.

### SUMMARY OF THE DISCLOSURE

The present disclosure is directed to a method and system for real-time detection and quantification of per- and polyfluoroalkyl substances (PFAS) using an ultra-fast electrochemical sensor (UP-FASt). In some embodiments, the UP-FASt can be composed of transition metals and/or graphene composites. In some embodiments, the system and method is a test kit system and method that can be utilized for electrochemical detection of PFAS in aqueous samples. In the kit system and method, the presence of PFOA or PFOS in the measurement solution can rely upon the interaction with the NiOOH electrocatalyst, producing a measurable change in the chronoamperometric current relative to a PFAS-free blank.

To address the need disclosed above, the present disclosure provides a field-deployable electrochemical sensing platform that enables rapid, ultra-trace detection of individual PFAS compounds and total PFAS in water and solid samples without extensive sample preparation. The present disclosure allows for sub-ppt detection limits, rapid analysis times of less than 30 seconds, and cost-effective sensor fabrication. The present disclosure provides a reusable sensor architecture with in-situ electrocatalyst regeneration, facilitating extended operational use. In certain embodiments, the electrochemical sensor integrates machine learning algorithms to enhance PFAS quantification and differentiation in complex environmental matrices.

Further, in accordance with an aspect of the present disclosure, a test kit system and method for the electrochemical detection of PFAS in an aqueous sample is provided. The test kit system includes: an activation solution comprising a hydroxide electrolyte at a first concentration; a blank solution comprising the hydroxide electrolyte at a second concentration and a volume of PFAS-free water, wherein the second concentration is achieved by dilution of a hydroxide stock solution with the PFAS-free water in a predetermined ratio; a sample solution vessel comprising the hydroxide stock solution at the same concentration as in the blank solution, and a remaining volume to be filled with the aqueous sample in the same predetermined ratio as the blank solution; and optionally, a cleaning solution comprising the hydroxide electrolyte.

In accordance with another aspect of the present disclosure the predetermined ratio of hydroxide stock solution to water is selected such that the final hydroxide concentration in both the blank and sample solutions is substantially the same, thereby ensuring that any difference in the electrochemical response between the blank and sample measurements is attributable to the presence of PFAS in the aqueous sample.

In general, in one embodiment, the present disclosure is directed to a system for detecting per- and polyfluoroalkyl substances (PFAS). The system can include a working electrode. The working electrode can include an electrode support. The system can include a catalyst. The system can also include a counter electrode. The counter electrode can include a material selected from the group consisting of platinum, gold, iridium, nickel, titanium, rhodium, ruthenium, and combinations thereof. The system can also include a reference electrode. The reference electrode can include a material selected from the group consisting of platinum, gold, silver, titanium, rhodium, ruthenium, mercury, mercury oxide, mercurous chloride, and combinations thereof. The system can also include a power source configured to apply a voltage between the working electrode and the reference electrode to facilitate electrochemical detection of PFAS.

In general, in another embodiment, the present disclosure is directed to a system for real-time electrochemical detection of per- and polyfluoroalkyl substances (PFAS). The system can include an electrochemical sensor having a working electrode, a counter electrode, and a reference electrode. The working electrode can include a transition metal or a graphene-metal composite. The system can also include a controller configured to regulate an applied voltage and measure electrochemical response. The system can also include a fluid handling system configured to transport a sample containing PFAS to the electrochemical sensor. The system can also include a data processing system configured to analyze the electrochemical response and determine PFAS concentration based on a calibration model.

In general, in another embodiment, the present disclosure is directed to a method for detecting per- and polyfluoroalkyl substances (PFAS) in a sample. The method can include inserting a sensor tip comprising a working electrode into an electrolyte solution or a sample containing PFAS. The method can also include applying a voltage between the working electrode and a reference electrode. The method can also include generating an electrocatalyst in situ at the working electrode through an electrochemical process. The method can also include measuring a change in current. The method can also include correlating the measured change in current to the concentration of PFAS in the sample.

In general, in another embodiment, the present disclosure is directed to a test kit system for the electrochemical detection of per- and polyfluoroalkyl substances (PFAS) in an aqueous sample. The test kit system includes an activation solution container including an activation solution including a hydroxide electrolyte at a first concentration suitable for cyclic voltammetric activation of a graphene-coated transition metal electrode. The test kit system further includes a blank solution container including a blank solution including the hydroxide electrolyte at a second concentration. The second concentration is achieved by dilution of a first hydroxide stock solution with PFAS-free water in a first predetermined volumetric ratio. The first hydroxide stock solution includes the hydroxide electrolyte. The test kit system further includes a sample solution container including a second hydroxide stock solution. The second hydroxide stock solution includes the hydroxide electrolyte. The second hydroxide stock solution has a volume and concentration in the sample solution container such that, by addition of the aqueous sample at a second predetermined volumetric ratio to the second hydroxide stock solution in the sample solution container, a sample solution is formed in which (i) the sample solution comprises the hydroxide electrolyte at the second concentration, and (ii) dilution factor of the PFAS-free water in the blank solution is equal to dilution factor of the aqueous sample in the sample solution.

In general, in another embodiment, the present disclosure is directed to a method for detecting PFAS in an aqueous sample using an above-described test kit system. The method includes activating a graphene-coated transition metal electrode by performing cyclic voltammetry utilizing the activation solution. The method further includes transferring the electrode to the blank solution. The method further includes allowing the electrode to rest at a first open circuit with rotation for at least 2 seconds. The method further includes measuring a first open circuit potential for at least 5 seconds. The method further includes performing a first chronoamperometry for at least 7 seconds. First current data in response to the first chronoamperometry is recorded as a function of time. The method further includes re-activating the graphene-coated transition metal electrode by performing cyclic voltammetry utilizing the activation solution or the blank solution. The method further includes transferring the electrode to the sample solution. The method further includes allowing the electrode to rest at a second open circuit with rotation for at least 2 seconds. The method further includes measuring a second open circuit potential for at least 5 seconds. The method further includes performing a second chronoamperometry for at least 7 seconds. Second current data in response to the second chronoamperometry is recorded as a function of time. The method further includes comparing the first current data to the second current data to detect PFAS in the aqueous sample.

### BRIEF DESCRIPTION OF THE DRAWINGS

Other advantages of the present disclosure will be apparent from the following detailed description of the disclosure in conjunction with embodiments as illustrated in the accompanying drawings, in which:

FIGS. 1A-1C depict a system for real-time detection and quantification of per- and polyfluoroalkyl substances integrating multiple transition metal-graphene electrocatalysts, controlled buffer systems, and deep learning algorithms for selective detection and quantification of regulated PFAS in water and solids, in accordance with certain embodiments of the present disclosure. FIG. 1A depicts an electrochemical sensor used in the system. FIG. 1B depicts the electrode tip used in electrochemical sensor shown in FIG. 1A. FIG. 1C depicts the system, including the electrochemical sensor shown in FIG. 1A.

FIG. 2 depicts a schematic of electrochemical cell for an UP-FASt sensor, in accordance with certain embodiments of the present disclosure.

FIGS. 3A-3C depict electrochemical characterizations comparing bare nickel and graphene-nickel composite electrode capabilities. FIG. 3A depicts cyclic voltammograms comparing bare nickel and nickel-graphene composite electrodes. FIG. 3B depicts Raman spectroscopy characterization confirming successful graphene integration and structural features of the composite, in accordance with certain embodiments of the present disclosure. FIG. 3C depicts chemical imaging of graphene-nickel composite electrodes.

FIGS. 4A-4C depict graphical data demonstrating the UP-FASt's capability to detect and quantify PFOA below EPA MCL values using DPV with an optimized cell configuration. FIG. 4A depicts DPV responses of nickel-graphene composite electrodes to varying PFOA concentrations in 0.1M KOH/0.12% v/v methanol electrolyte, in accordance with certain embodiments of the present disclosure. FIG. 4B depicts a chart of concentration versus signal, in accordance with certain embodiments of the present disclosure. FIG. 4C depicts a linear calibration curve correlating maximum differential current with PFOA concentration, in accordance with certain embodiments of the present disclosure.

FIGS. 5A-5C depict graphical data demonstrating the results of utilizing an electrochemical cell for the UP-FASt sensor, in accordance with certain embodiments of the present disclosure. FIG. 5A depicts a DPV response using bare nickel foil as a working electrode, in accordance with certain embodiments of the present disclosure. FIG. 5B depicts current density during cyclic voltammetry of the bare nickel working electrode. FIG. 5C depicts current density during cyclic voltammetry of the nickel-graphene working electrode.

FIGS. 6A-6C depict a scheme for a sensor methodology analyzing PFOA in water, in accordance with certain embodiments of the present disclosure. FIG. 6A depicts a protocol utilizing the UP-FASt sensor for PFOA detection. FIGS. 6B-6C depict, respectively a baseline current and test sample current that are recorded during the protocol shown in FIG. 6A.

FIGS. 7A-7C depict a signal analysis methodology for PFOA detection using the nickel-graphene composite electrode, in accordance with certain embodiments of the present disclosure. FIG. 7A depicts a quantification method based on differential current maxima between sample and baseline signals, in accordance with certain embodiments of the present disclosure. FIG. 7B depicts a peak potential shift correlating with PFOA concentration, in accordance with certain embodiments of the present disclosure. FIG. 7C depicts development of characteristic signal features at elevated concentrations, in accordance with certain embodiments of the present disclosure.

FIG. 8 depicts structure of PFAS molecules regulated by the EPA, in accordance with certain embodiments of the present disclosure.

FIGS. 9A-9D depict four reagent solutions for a test kit system (10 mL configuration for each component), in accordance with certain embodiments of the present disclosure.

FIGS. 10A-10D depict four reagent solutions for a test kit system (5 mL configuration for each component), in accordance with certain other embodiments of the present disclosure.

FIGS. 11A-11B depict volume ratios and dilution matching for blank and sample preparation, respectively, in accordance with certain embodiments of the present disclosure.

FIG. 12 depicts a PFAS test workflow using a test kit, in accordance with certain other embodiments of the present disclosure.

### NOTATION AND NOMENCLATURE

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or a direct connection. Thus, if a first device couples to a second device, that connection may be through a direct connection or through an indirect connection via other devices and connections.

The terminology used herein is for the purpose of describing particular example embodiments only, and is not intended to be limiting. Following long-standing patent law convention, the terms “a” and “an” mean “one or more” when used in this application, including the claims.

As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer, or section from another region, layer, or section. Terms such as “first,” “second,” and other numerical terms, when used herein, do not imply a sequence or order unless clearly indicated by the context. Thus, a first element, component, region, layer, or section discussed below could be termed a second element, component, region, layer, or section without departing from the teachings of the example embodiments. The phrase “at least one of,” when used with a list of items, means that different combinations of one or more of the listed items may be used, and only one item in the list may be needed. As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D. Accordingly, as an example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Spatially relative terms, such as “inner,” “outer,” “beneath,” “below,” “lower,” “above,” “upper,” “top,” “bottom,” and the like, may be used herein. These spatially relative terms can be used for ease of description to describe one element's or feature's relationship to another element(s) or feature(s) as illustrated in the figures. The spatially relative terms may also be intended to encompass different orientations of the device in use, or operation, in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptions used herein interpreted accordingly.

Unless otherwise indicated, all numbers expressing quantities of ingredients, reaction conditions, and so forth used in the specification are to be understood as being modified in all instances by the term “about.” Accordingly, unless indicated to the contrary, the numerical parameters set forth in this specification are approximations that can vary depending upon the desired properties sought to be obtained by the presently disclosed subject matter.

### DETAILED DESCRIPTION OF THE DISCLOSURE

The present disclosure provides systems and methods for real-time electrochemical detection and quantification of per- and polyfluoroalkyl substances (PFAS). Further, the present disclosure provides a system and method for detecting per- and polyfluoroalkyl substances (PFAS) using an electrochemical sensor. The present disclosure further provides for a test kit system and method that can be utilized for electrochemical detection of PFAS in aqueous samples.

The system can include a working electrode, a counter electrode, and a reference electrode, along with a power source for applying voltage to facilitate electrochemical detection. The system and method are designed to enable real-time, field-deployable detection of PFAS in various liquid and solid samples with high sensitivity and selectivity.

In some embodiments, the system comprises a working electrode. The working electrode includes an electrode support and a catalyst. The electrode support may be composed of a conductive material, which allows for efficient electron transfer and ensures durability under operational conditions. In certain embodiments, the electrode support is selected from the group consisting of nickel, copper, titanium, zinc, stainless steel, Hastelloy, graphite, and aluminum. The electrode support may be provided in the form of foil, sheet, plate, gauze, mesh, foam, or a combination thereof to optimize surface area and reactivity.

In some embodiments, the catalyst of the working electrode comprises a transition metal. The transition metal may be selected from the group consisting of nickel, iron, zinc, copper, cobalt, chromium, molybdenum, rhodium, ruthenium, palladium, iridium, and combinations thereof. In some implementations, the catalyst comprises a composite of graphene and at least one metal, enhancing the electrocatalytic activity for PFAS detection. In certain configurations, the electrode support and the catalyst are the same material, such that the working electrode itself serves as both a conductive base and an active electrocatalyst. When the electrode support and catalyst are the same, they may be selected from nickel, iron, cobalt, chromium, copper, molybdenum, rhodium, ruthenium, palladium, iridium, and combinations thereof.

The system also includes a counter electrode, which functions to complete the electrochemical circuit. The counter electrode may be composed of a material selected from the group consisting of platinum, gold, iridium, nickel, titanium, rhodium, ruthenium, and combinations thereof. In some embodiments, the counter electrode may include the same material as the anode.

A reference electrode is also included in the system to provide a stable reference potential. The reference electrode may be composed of a material selected from the group consisting of platinum, gold, silver, titanium, rhodium, ruthenium, mercury, mercury oxide, mercurous chloride, and combinations thereof.

In some embodiments, the system includes a power source configured to apply a voltage between the working electrode and the reference electrode. The applied voltage facilitates electrochemical detection of PFAS by generating redox reactions that allow for quantification of PFAS compounds.

In certain embodiments, the system is further configured for real-time electrochemical detection of PFAS. The system may include a controller that regulates the applied voltage and measures the electrochemical response. A fluid handling system may be provided to transport a sample containing PFAS to the electrochemical sensor, facilitating continuous or batch-wise analysis of environmental or industrial samples. A data processing system may also be included to analyze the electrochemical response and determine PFAS concentration based on a calibration model, allowing for automated quantification and detection.

In some embodiments, the working electrode of the electrochemical sensor includes an electrode support and a catalyst, as described above. The electrode support comprises a conductive material, which may be selected from nickel, copper, titanium, zinc, stainless steel, Hastelloy, graphite, and aluminum. The electrode support may also be provided in the form of foil, sheet, plate, gauze, mesh, foam, or a combination thereof. The catalyst of the working electrode may comprise a transition metal, such as nickel, iron, zinc, copper, cobalt, chromium, molybdenum, rhodium, ruthenium, palladium, iridium, or combinations thereof. In other embodiments, the catalyst comprises a composite of graphene and at least one metal, which provides enhanced surface interactions and improved electrochemical detection.

In some implementations, the electrode support and catalyst are the same material, selected from nickel, iron, cobalt, chromium, copper, molybdenum, rhodium, ruthenium, palladium, iridium, and combinations thereof. The counter electrode may be composed of platinum, gold, iridium, nickel, titanium, rhodium, ruthenium, and combinations thereof. The reference electrode may be composed of platinum, gold, silver, titanium, rhodium, ruthenium, mercury, mercury oxide, mercurous chloride, and combinations thereof.

The present disclosure further provides a method for detecting PFAS in a sample. The method can include inserting a sensor tip, which includes a working electrode, into an electrolyte solution or a sample containing PFAS. A voltage is applied between the working electrode and a reference electrode to facilitate detection. The applied voltage may range from −0.5 V to 1 V versus the reference electrode, depending on the composition of the electrodes and the target PFAS compounds.

The method includes generating an electrocatalyst in situ at the working electrode through an electrochemical process. This process allows for the regeneration of the electrocatalyst, ensuring long-term sensor usability and minimizing operational costs. In some embodiments, the catalyst can be electrochemically created locally on the sensor tip of the working electrode by applying a voltage window between the working electrode and the reference, depending on the electrodes.

The method further can include measuring a change in current, which correlates to the concentration of PFAS in the sample. The change in current may be measured using differential pulse voltammetry (DPV) or chronoamperometry (CA). In some embodiments, the applied voltage varies depending on factors such as compounds of interest and the composition of the working electrode. Further, in some embodiments, the applied potential varies from −0.5 to 1 V versus reference electrode depending on the working and reference electrodes used. These electrochemical techniques allow for rapid, sensitive detection of PFAS in complex matrices.

The electrolyte solution used in the method may comprise at least one of potassium hydroxide (KOH), sodium hydroxide (NaOH), ammonium solutions, phosphate buffers, or a combination thereof. In some embodiments, the electrolyte solution includes at least one organic solvent, such as methanol, ethanol, isopropanol, or acetone, which may enhance PFAS detection by modifying the electrode interface. The electrolyte solution may have a pH between 9.5 and 14, which provides optimal conditions for electrochemical analysis.

The sensor tip may be inserted into the electrolyte solution or the sample directly, depending on the nature of the sample being analyzed. In certain embodiments, the sensor tip rotates at a speed between 0 and 3000 rpm, which may enhance mass transport and improve detection sensitivity.

The method further includes controlling the temperature of the system within a range of 10° C. to 40° C. Temperature control ensures stability and reproducibility of electrochemical measurements, particularly in field applications where environmental fluctuations may impact sensor performance.

The liquid extracted from biosolids (for example, but not limited to manure or sludge) or solid samples (for example, but not limited to soil samples) can be used to determine PFAS in solid samples. In such embodiments, the biosolids or solid samples are shaken with the solvent and the supernatant liquid is separated and used as the sample. In some embodiments, the solvent includes buffer, methanol, ethanol, isopropanol, acetone, or combinations thereof.

### Systems and Methods

The system and method described herein provide significant advantages over conventional PFAS detection techniques. The real-time electrochemical sensing approach enables rapid detection at ultra-trace levels, eliminates the need for complex sample preparation, and offers a cost-effective alternative to mass spectrometry-based methods. The ability to perform on-site PFAS detection with a field-deployable electrochemical sensor represents a critical advancement in environmental monitoring and regulatory compliance.

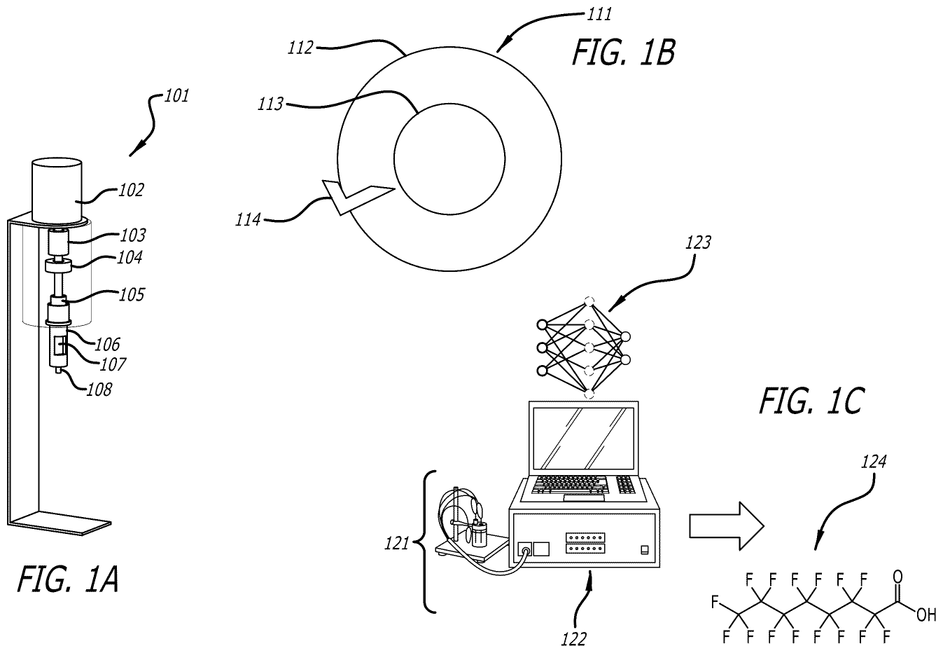

FIGS. 1A-1C depict a system for real-time detection of PFAS integrating multiple transition metal-graphene electrocatalysts, controlled buffer systems, and deep learning algorithms for selective detection and quantification of regulated PFAS in water and solids, in accordance with certain embodiments of the present disclosure.

In particular, FIGS. 1A-1C illustrate an ultra-fast electrochemical sensor architecture for real-time detection and quantification of per- and polyfluoroalkyl substances (PFAS) in water and solids. The electrochemical sensor 101 can integrate multiple transition metal-graphene electrocatalysts, controlled buffer systems, and deep learning algorithms 123 to enable selective detection of PFAS compounds 124. The system is designed to operate in complex environmental matrices, achieving ultra-trace detection limits while maintaining rapid response times.

As depicted in FIG. 1A, the electrochemical sensor 101 includes a DC motor 102, plastic coupling 103, bearings 104-105, a replaceable body tip 106, working electrode shaft 107 (with a working electrode surface), and an insulator sleeve 108.

As depicted in FIGS. 1A-1B, the electrochemical sensor 101 utilizes transition metal-graphene composite working electrodes (with electrode tip 111), which may include nickel (Ni), copper (Cu), zinc (Zn), cobalt (Co), iron (Fe), or combinations thereof, to enhance electrocatalytic activity and facilitate PFAS detection. In some embodiments and as shown in FIG. 1B, the electrode tip 111 may be configured with a rotating disk electrode (such as transition metal-graphene electrode (working electrode) 113 with a Pt ring (counter electrode) 112 and Pt foil (reference electrode) 114 to improve mass transport and enable discrimination of different PFAS compounds.

As depicted in FIG. 1C, the system further incorporates a controlled buffer system 121 to mitigate environmental interferences and maintain optimal electrochemical conditions. This is coupled to the electronic and mini-potentiostat 122.

The sensor architecture also supports the implementation of deep learning algorithms 123 for signal processing, enabling characterization and quantification of PFAS based on distinct electrochemical signatures. In certain embodiments, the sensor integrates a machine learning-based classification model to differentiate between multiple PFAS compounds in a single sample. The versatility of the platform allows for the use of multiple electrode materials and calibration models, adapting to various environmental conditions.

The sensor unit can be designed to be compact and portable, with dimensions comparable to standard handheld pH meters, facilitating field deployment. The system can be utilized in groundwater monitoring wells, industrial discharge points, wastewater treatment facilities, and other locations where real-time PFAS detection is critical. I.e., the system can be used for the rapid quantification of PFAS in groundwater.

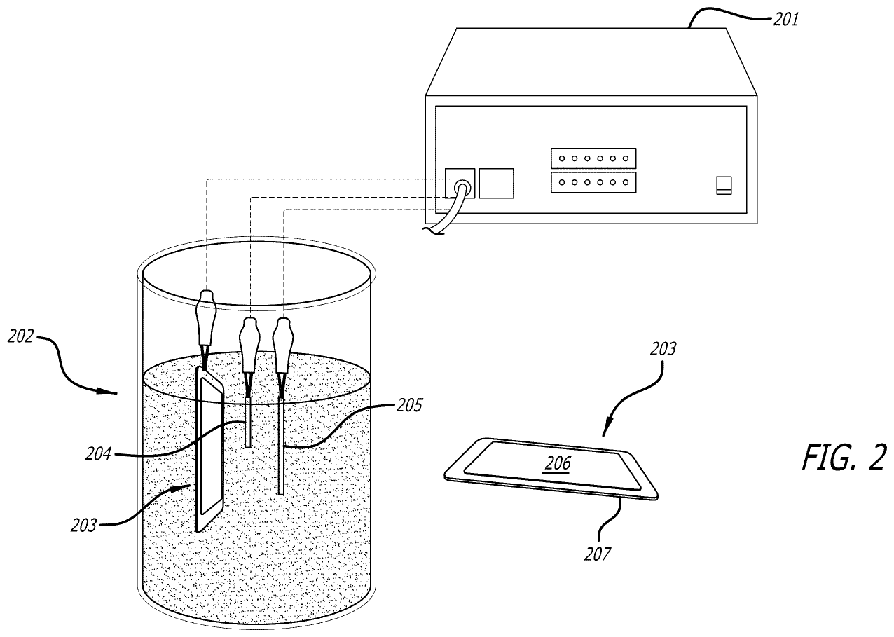

FIG. 2 depicts a schematic of an electrochemical cell 202 for an UP-FASt sensor, in accordance with certain embodiments of the present disclosure. The UP-FASt sensor includes a three-electrode electrochemical glass cell designed to be PFAS-free and connected to potentiostat 201 (such as a Gamry 1010 Potentiostat/Galvanostat/ZRA). The setup incorporates a nickel-graphene nanocomposite working electrode 203, a platinum foil counter electrode 204, and a platinum wire pseudo-reference electrode 205. The electrochemical system is designed to facilitate real-time PFAS detection with high specificity and reproducibility.

As shown in FIG. 2, the working electrode 203 was a nickel-graphene nanocomposite (nickel 207 and graphene 206) with dimensions of approximately 0.7 cm×0.7 cm and a thickness of 0.1 mm, providing an exposed area of 0.49 cm². The counter electrode 205 was a platinum foil measuring approximately 1.0 cm×0.7 cm with a thickness of 0.1 mm. A platinum wire pseudo-reference electrode 204 (0.5 mm diameter, 1 cm length) was positioned between the working and counter electrodes to provide a stable reference potential. The electrochemical measurements were conducted in a PFAS-free glass cell with a total volume of 25 mL, containing a solution volume of 16 mL (0.1 mL sample+15.9 mL electrolyte), maintained at a controlled temperature of 25±0.1° C.

The electrolyte solution consisted of 0.1 M potassium hydroxide (KOH) with 0.12% v/v methanol. The use of methanol serves two primary functions: ensuring consistency with standard reference samples and enhancing PFAS-electrode interactions through increased hydrophobicity at the electrode interface. Sample preparation for electrochemical testing involved serial dilution of perfluorooctanoic acid (PFOA) solutions, which were prepared from a 50 ppm stock solution using PFOA powder (Sigma Aldrich, catalog number 171468). Working standards at 1, 2, and 4 ppt were prepared by diluting the stock solution with 0.1 M KOH containing 0.12% v/v methanol. All solutions were prepared in PFAS-free glassware with pre-tested PFAS-free reagents to prevent contamination.

The nickel-graphene nanocomposite working electrode 203 was fabricated using a chemical vapor deposition (CVD) process, where graphene was directly grown on the nickel foil. In other environments the graphene could be grown by CVD on Cu foil, and then removed and transferred to the Ni foil. The optimized electrode architecture enhances PFAS detection through a combination of electrochemical and adsorption-based mechanisms. The composite structure was designed to maximize surface interactions, facilitate rapid electron transfer, and provide a stable electrocatalytic platform for ultra-trace PFAS quantification.

The electrochemical detection mechanism of the UP-FASt sensor is based on the electrochemical transformation of transition metal oxyhydroxide (Mⁿ⁺OOH) species, as represented by the redox reaction:

$${Ni ( OH )}_{2} ( s ) + {OH}^{-} ⇌ NiOOH ( s ) + {H}_{2} O ( l ) + {e}^{-} \qquad ( 1 )$$

This redox process can serve as the foundation for PFAS quantification, wherein the analyte modulates the electrochemical response through specific interactions with the metal oxyhydroxide surface species. The sensor configuration, as shown in FIG. 2, allows for highly selective and reproducible PFAS detection without the need for extensive sample preparation or specialized reagents.

The sensor achieved sub-ppt detection limits through a combination of optimized electrode composition, electrochemical signal processing, and controlled buffer conditions. The incorporation of transition metal-graphene composites (e.g., Ni, Cu, Zn, Co, Fe, and their combinations) further enhances electrocatalytic activity, improving both sensitivity and selectivity. The UP-FASt sensor was designed to operate in a simple KOH/methanol buffer system, eliminating interference from complex sample matrices and enabling real-time monitoring in environmental applications.

### Electrochemical Characterizations

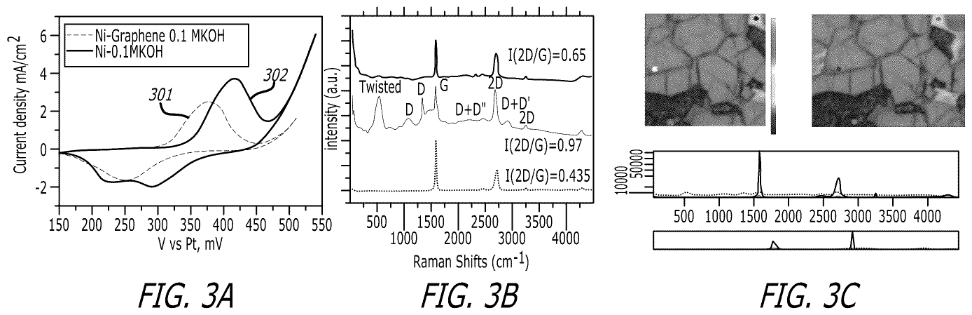

FIGS. 3A-3C depict electrochemical characterizations comparing bare nickel and graphene-nickel composite electrode capabilities. FIG. 3A depicts cyclic voltammograms comparing bare nickel and nickel-graphene composite electrodes in 0.1M KOH, demonstrating enhanced electrocatalytic activity of the composite, in accordance with certain embodiments of the present disclosure. FIG. 3B depicts Raman spectroscopy characterization confirming successful graphene integration and structural features of the composite, in accordance with certain embodiments of the present disclosure. FIG. 3C depicts chemical imaging of the graphene-nickel composite electrodes.

FIGS. 3A-3C illustrate the electrochemical characterizations comparing a bare nickel electrode with a nickel-graphene composite electrode, demonstrating the enhanced electrocatalytic activity and structural advantages of the composite, in accordance with certain embodiments of the present disclosure.

FIG. 3A (with plots 301-302 for Ni-graphene 0.1M KOH and Ni- 0.1M KOH, respectively) depicts cyclic voltammograms obtained in 0.1M KOH for both bare nickel and nickel-graphene composite electrodes, revealing significant improvements in electrochemical performance for the composite electrode. As shown in FIG. 3A, the nickel-graphene composite exhibits reduced overpotential, indicative of enhanced electron transfer kinetics, and superior electrocatalytic stability compared to the bare nickel electrode. Electrochemical conditioning of the electrodes was conducted to develop stable Ni(OH)₂/NiOOH active phases, with the bare nickel electrode requiring approximately 1,500 conditioning cycles, whereas the nickel-graphene composite stabilized within 150 cycles, demonstrating a significantly faster equilibration process. These results indicate that the composite architecture facilitates rapid catalyst activation and provides a stable electrochemical interface suitable for sensing applications.

FIG. 3B presents Raman spectroscopy characterization confirming the successful integration of graphene within the nickel-graphene composite. The presence of characteristic graphene spectral features, including the D, G, and 2D bands, indicates the formation of a structurally robust graphene layer. The defect-engineered few-layer graphene deposited on transition metal surfaces, such as nickel, enhances both the material's conductivity and its ability to regulate molecular and ionic transport at the electrode interface. In certain embodiments, the graphene layer is grown directly on nickel via chemical vapor deposition (CVD) or transferred post-synthesis from copper substrates. This engineered interface enables precise molecular gating, regulating the transport of analytes and ionic species (e.g., OH⁻, H⁺) to the active sites of the electrode.

FIG. 3C depicts chemical imaging of nickel-graphene composite electrodes. The composite electrode exhibits a highly uniform surface morphology with an optimized nanostructure, which promotes enhanced analyte adsorption and selective charge transfer processes. The engineered interface of the composite electrode enables suppression of competing electrochemical reactions, such as oxygen evolution, while maintaining high sensitivity for target analytes.

The electrochemical sensing platform described herein leverages a combination of electrochemical and adsorption-based mechanisms to achieve selective PFAS detection. The nickel-graphene composite electrode architecture provides distinct advantages, including controlled molecular and ionic transport optimization, which enhances catalyst formation and charge transfer kinetics; and active site protection, which prevents surface poisoning and ensures sustained electrocatalytic activity.

The composite electrode's capability for detecting non-electroactive PFAS molecules is attributed to three primary synergistic interactions: (1) the electrocatalytic activity of the Mⁿ⁺OOH surface species, (2) non-covalent adsorption forces—including hydrophobic interactions, π-π stacking, and van der Waals forces—between PFAS molecules and the graphene surface, and (3) optimized mass transport properties of the composite architecture. In some embodiments, the electrode structure enables molecular recognition of specific PFAS functional groups, such as carboxyl (—COOH) and sulfonic (—SO₃H) groups, enhancing its selectivity for different PFAS compounds.

The nickel-graphene composite electrode, as characterized in FIGS. 3A-3C, demonstrates significant improvements in electrochemical performance, selectivity, and stability, making it well-suited for real-time detection and quantification of PFAS in environmental samples.

### Capability to Detect and Quantify

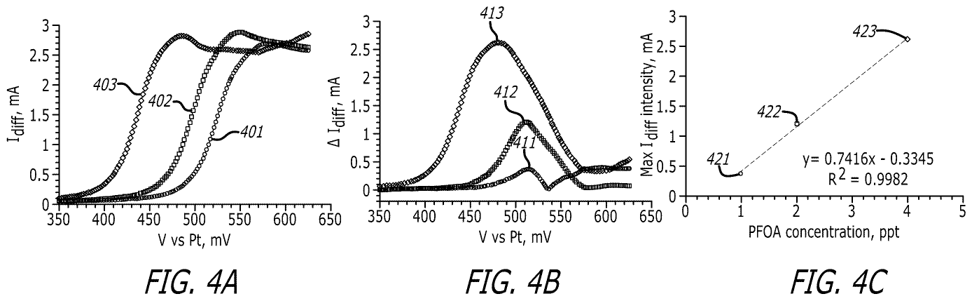

FIG. 4A-4C depict graphical data demonstrating the UP-FASt's capability to detect and quantify PFOA below EPA MCL values using DPV with an optimized cell configuration. FIG. 4A depicts DPV responses of nickel-graphene composite electrodes to varying PFOA concentrations in 0.1M KOH/0.12% v/v methanol electrolyte, in accordance with certain embodiments of the present disclosure. FIG. 4B depicts a chart of concentration versus signal, in accordance with certain embodiments of the present disclosure. FIG. 4C depicts a linear calibration curve correlating maximum differential current with PFOA concentration, in accordance with certain embodiments of the present disclosure.

FIGS. 4A-4C depict graphical data demonstrating the capability of the UP-FASt sensor to detect and quantify perfluorooctanoic acid (PFOA) at concentrations below the maximum contaminant levels (MCLs) set by the United States Environmental Protection Agency (EPA), using differential pulse voltammetry (DPV) with an optimized electrochemical cell configuration.

FIG. 4A (with plots 401-403 for 1 ppt, 2 ppt, and 4 ppt, respectively) illustrates the DPV responses of nickel-graphene composite electrodes exposed to varying PFOA concentrations in a 0.1M KOH/0.12% v/v methanol electrolyte solution. As shown, the nickel-graphene composite electrode exhibits distinct, concentration-dependent responses, demonstrating the ability to resolve ultra-trace levels of PFOA. The electrochemical response is characterized by differential peak currents that correlate with PFOA concentration, establishing a direct quantitative relationship.

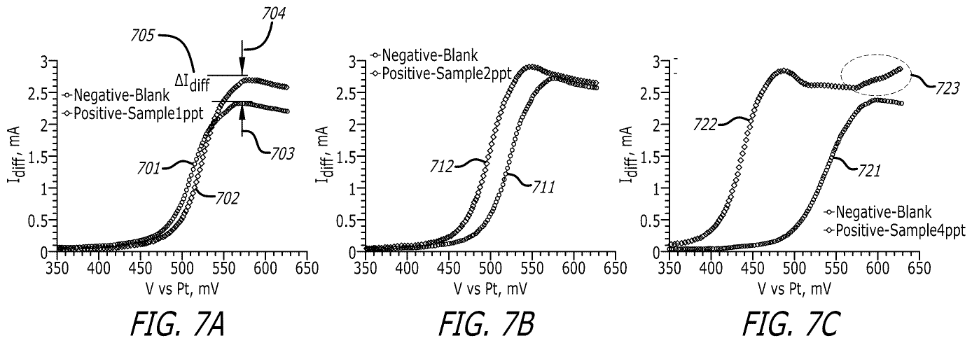

FIG. 4B (with plots 411-413 for 1 ppt, 2 ppt, and 4 ppt, respectively) depicts a chart plotting signal intensity as a function of PFOA concentration. Dynamic baseline subtraction techniques, as implemented in FIGS. 7A-7C (described and discussed below), enhance signal resolution, enabling precise quantification of PFOA concentrations. The electrochemical responses exhibit reproducible peak positions and current intensities, further confirming the system's sensitivity and robustness.

FIG. 4C depicts a linear calibration curve correlating the maximum differential current (ΔI_{diff}) with PFOA concentration, demonstrating an exceptional linear relationship (R²>0.99). See FIG. 4C (with points 421-423 for 1 ppt, 2 ppt, and 4 ppt, respectively, having a linear relationship y=0.7416x−0.3345 (R²=0.9982)). This calibration model establishes a reproducible method for PFAS quantification. The well-defined correlation between electrochemical signals and analyte concentrations provides a basis for developing machine learning algorithms capable of optimizing detection accuracy and selectivity. In some embodiments, multiple electrochemical signal features—including peak position, current intensity, and waveform characteristics—serve as inputs for machine learning-based PFAS differentiation, as described below in FIGS. 7A-7C.

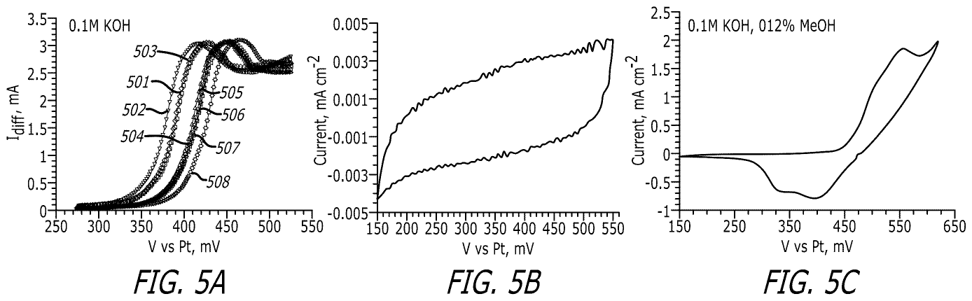

As demonstrated in FIGS. 5A-5C, bare nickel electrodes in the absence of the appropriate buffer fail to achieve quantifiable electrochemical responses at sub-ppt PFOA concentrations, highlighting the role of the engineered graphene-metal composite electrode architecture in enabling ultra-trace detection. The nanostructured graphene interface enhances electrochemical interactions through optimized charge transfer properties, selective molecular adsorption, and improved electrocatalytic activity.

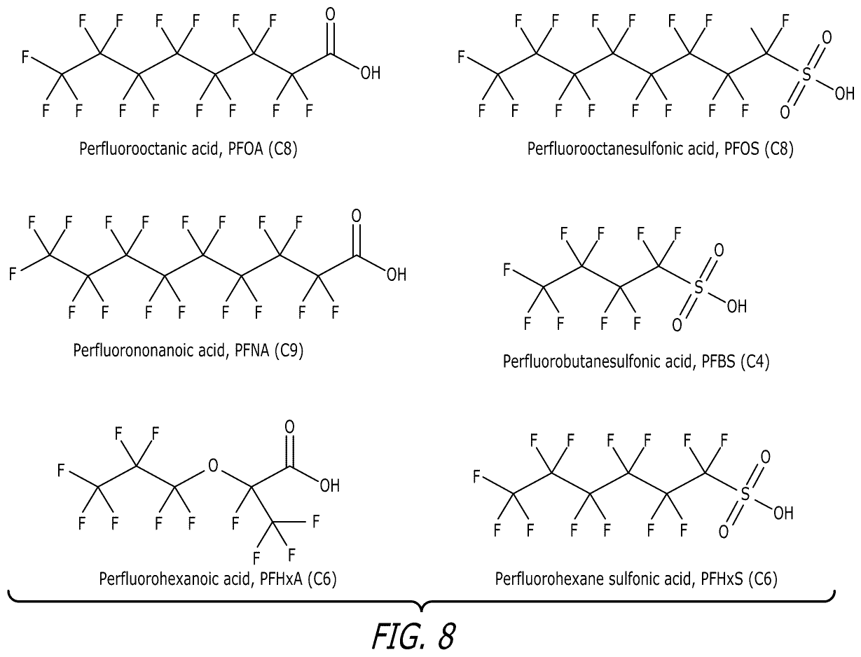

The results shown in FIGS. 4A-4C establish a foundation for broader PFAS detection using the UP-FASt platform. While initial testing focused on PFOA, the graphene-composite electrode architecture facilitates non-specific interactions, including hydrophobic forces, T-T stacking, and van der Waals interactions, making it applicable for detecting a range of PFAS compounds sharing similar functional groups (for example, but not limited to carboxylic and sulfonic acids), as illustrated in FIG. 8 (discussed below).

The UP-FASt platform overcomes key limitations of existing PFAS detection technologies, including insufficient detection limits, long analysis times, and extensive sample preparation requirements. The electrochemical detection approach eliminates the need for complex pretreatment steps, while enabling real-time, field-deployable quantification of PFAS in diverse environmental matrices.

The electrochemical sensing principle of the UP-FASt sensor is based on selective interactions between target PFAS analytes and electroactive metal oxyhydroxide (Mⁿ⁺OOH) surface species. The detection mechanism relies on measurable modulations in the redox current of Mn+OOH species, which are influenced by PFAS adsorption at the electrode-electrolyte interface. In an alkaline medium, the metal undergoes an electrochemical transition, as described by reaction (1), above.

The presence of PFAS at the electrode-electrolyte interface modulates the kinetics of this redox transformation, resulting in detectable electrochemical signatures that enable real-time PFAS quantification. The engineered composite electrode architecture enhances these interactions, facilitating both selective adsorption and rapid electron transfer processes.

As demonstrated in FIGS. 4A-4C, the UP-FASt sensor achieves sub-ppt detection limits, rapid response times (<30 seconds per measurement), and robust reproducibility across different test conditions. The integration of deep learning-based signal processing, as further detailed in FIG. 1C, offers additional advantages by enhancing compound selectivity and mitigating matrix interferences.

The electrochemical characterization results presented in FIGS. 4A-4C validate the UP-FASt platform as an effective and scalable solution for PFAS monitoring in environmental samples.

### UP-FASt Sensor

FIGS. 5A-5C depict graphical data demonstrating the results of utilizing an electrochemical cell for the UP-FASt sensor, in accordance with certain embodiments of the present disclosure. FIG. 5A depicts a DPV response using bare nickel foil as a working electrode, in accordance with certain embodiments of the present disclosure. FIG. 5B depicts current density during cyclic voltammetry of the bare nickel working electrode. FIG. 5C depicts current density during cyclic voltammetry of the nickel-graphene working electrode.

FIGS. 5A-5C demonstrate the performance differences between bare nickel and nickel-graphene composite electrodes in the electrochemical detection of PFOA. As shown in FIG. 5A, initial experiments utilizing the electrochemical cell configuration failed to provide a quantifiable DPV response to PFOA at ppb-level concentrations when using bare nickel as the working electrode in the absence of methanol. FIG. 5A (with plots 501-508 for blank, 5 ppb, 15 ppb, 25 ppb, 35 ppb, 50 ppb, 70 ppb, and 100 ppb, respectively). The lack of detectable electrochemical response reveals that bare nickel alone does not exhibit sufficient electrocatalytic activity or adsorption capacity to facilitate ultra-trace PFOA detection.

The absence of a significant DPV response in FIG. 5A is hypothesized to be influenced by the lack of methanol in the electrolyte solution. Methanol has been observed to mediate electrochemical interactions through potential coordination effects and surface interaction mechanisms that may enhance PFAS adsorption at the electrode-electrolyte interface. The role of methanol in improving detection sensitivity is further investigated through variations in methanol concentration to optimize sensor performance while maintaining measurement stability.

FIG. 5B presents cyclic voltammetry (CV) results obtained using a bare nickel working electrode. The data illustrate the depletion of the nickel oxyhydroxide (NiOOH) active phase over successive electrochemical cycles, which is indicative of the limited stability of the bare nickel electrode during repeated measurements. The loss of active NiOOH species reduces the sensor's ability to facilitate consistent redox reactions necessary for reliable PFAS quantification.

FIG. 5C presents cyclic voltammetry results obtained using the nickel-graphene composite working electrode. Unlike the bare nickel electrode, the nickel-graphene composite maintains a stable NiOOH active phase over successive cycles, suggesting improved electrochemical durability and sensor longevity. The sustained presence of NiOOH in FIG. 5C, compared to its depletion in FIG. 5B, indicates that the graphene composite structure enhances stability by mitigating catalyst degradation and surface passivation effects.

The differences observed in FIGS. 5A-5C highlight the advantages of the nickel-graphene composite electrode architecture in the UP-FASt sensor. The composite structure provides enhanced electrocatalytic activity, improved stability of the NiOOH active phase, and superior charge transfer properties compared to bare nickel. These factors contribute to the system's ability to achieve ultra-trace PFAS detection at ppt-level concentrations, as demonstrated in FIGS. 4A-4C.

The data presented in FIGS. 5A-5C support the selection of nickel-graphene composite electrodes as optimal working electrode materials for the UP-FASt sensor. The incorporation of graphene into the electrode structure enhances performance by improving electrochemical kinetics, increasing active surface area, and stabilizing the electrocatalytic phase required for PFAS quantification.

### Sensor Methodology

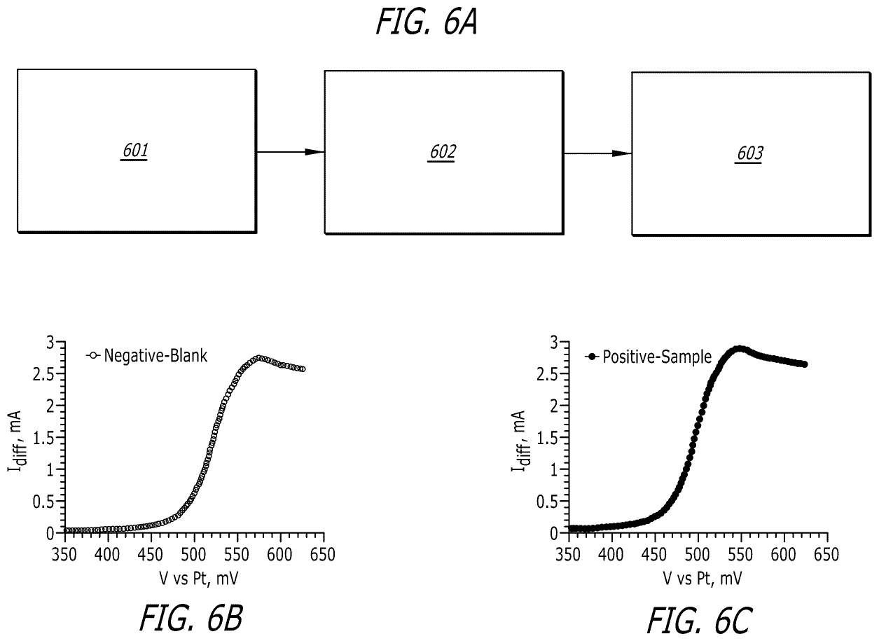

FIGS. 6A-6C depict a scheme for a sensor methodology analyzing PFOA in water, in accordance with certain embodiments of the present disclosure.

FIG. 6A illustrates an experimental protocol utilized by the UP-FASt sensor for PFOA detection, employing differential pulse voltammetry (DPV) as the primary electrochemical technique. The electrochemical cell configuration used in the analysis is shown in FIG. 2 and discussed above (with the electrochemical detection mechanism based on redox reaction (1)).

As depicted in FIG. 6A, step 601 is to record current in the baseline. This can be done by applying potential versus a reference sample, such as for 12 seconds, and then recording current. FIG. 6B depicts a baseline current recorded. Step 602 is to record current in the test sample. This can be done by applying potential versus a reference sample, such as for 12 seconds, and then recording current. FIG. 6C depicts a test sample current recorded. Step 603 is a rinse step, such as immersion in 0.1 KOH for 10 seconds, and then proceeding back to step 601. Such a full processing of a sample can then take less than a minute.

The DPV parameters were optimized to enable rapid detection of PFOA, achieving a total measurement time of less than one minute per sample. The optimized DPV conditions include an initial potential of 0.35 V, a final potential of 0.62 V versus the platinum reference electrode, a potential increment of 0.002 V, a potential amplitude of 0.075 V, a pulse width of 0.05 seconds, and a pulse period of 0.08 seconds. These parameters were selected to enhance sensitivity and ensure reproducible quantification of PFOA at ultra-trace concentrations.

The analytical procedure implements a dynamic baseline measurement approach, wherein a blank electrolyte solution (0.1 M KOH containing 0.12% v/v methanol) is analyzed before each sample measurement. This baseline measurement enables real-time signal validation, background subtraction, and drift correction, thereby improving accuracy and reproducibility. The use of a dynamic baseline methodology allows for compensation of potential interferences arising from electrolyte composition and environmental variations.

The dynamic baseline methodology ensures high-quality signal acquisition and standardization of measurement conditions across different samples. The approach provides enhanced robustness in electrochemical signal processing, as further demonstrated in FIGS. 7A-7C. The rapid testing capability and cost-effectiveness of the baseline solution composition make this method particularly suitable for real-time, field-deployable PFAS monitoring applications.

FIG. 7A-7C depict a signal analysis methodology for PFOA detection using the nickel-graphene composite electrode, in accordance with certain embodiments of the present disclosure. FIG. 7A depicts a quantification method based on differential current maxima between sample and baseline signals, in accordance with certain embodiments of the present disclosure. FIG. 7B depicts a peak potential shift correlating with PFOA concentration, in accordance with certain embodiments of the present disclosure. FIG. 7C depicts development of characteristic signal features at elevated concentrations, in accordance with certain embodiments of the present disclosure.

As shown in FIG. 7A (1 ppt PFOA concentration with plots 701-702 for negative-blank and positive-sample 1 ppt, respectively), the quantification methodology implemented in the UP-FASt sensor platform relies on differential pulse voltammetry (DPV) measurements, wherein the differential current maxima 705 (ΔI_{diff}) between sample signals (sample max current 704 of plot 702) and baseline signals (baseline max current 703 of plot 701) serve as a primary analytical metric. This differential current-based approach provides a robust and reproducible means of quantifying ultra-trace levels of PFOA in complex environmental matrices.

FIG. 7B (2 ppt PFOA concentration with plots 711-712 for negative-blank and positive-sample 1 ppt, respectively) demonstrates that increasing PFOA concentration induces a peak potential shift, indicating systematic variations in electrochemical response as a function of analyte concentration. (The potential for sample max current decreased with the increasing PFOAs concentration.) This shift is associated with changes in adsorption dynamics and electrostatic interactions at the nickel-graphene electrode interface. The ability to detect such peak shifts enhances the platform's capability for distinguishing between different PFAS compounds, as peak position and shape may serve as unique electrochemical fingerprints for various analytes.

FIG. 7C (4 ppt PFOA concentration with plots 721-722 for negative-blank and positive-sample 1 ppt, respectively) illustrates the development of additional characteristic signal features (such as tail feature 723) at higher PFOA concentrations. These features include variations in peak width, signal intensity, and waveform characteristics, which provide multiple parameters for analyte identification and quantification. The emergence of such complex signal structures suggests potential for deep learning applications, where convolutional neural networks (CNNs) and pattern recognition algorithms can be implemented to improve selectivity and sensitivity in detecting and differentiating multiple PFAS species.

The signal processing architecture of the UP-FASt sensor enables the incorporation of multiple electrocatalysts, each tailored to specific PFAS functional groups (-carboxylic, -sulfonic). By leveraging advanced signal analysis methodologies, including dynamic baseline correction, as shown in FIG. 6, and multi-parameter electrochemical characterization, as shown in FIGS. 4A-4C, the sensor provides a scalable platform for real-time PFAS quantification in field environments. The ability to process distinct electrochemical signatures across varying PFAS concentrations enhances the detection accuracy of the system while maintaining ultra-trace detection limits.

FIG. 8 depicts the molecular structures of per- and polyfluoroalkyl substances (PFAS) that are regulated by the United States Environmental Protection Agency (EPA), in accordance with certain embodiments of the present disclosure. The illustrated PFAS compounds include perfluorooctanoic acid (PFOA), perfluorooctane sulfonic acid (PFOS), perfluorononanoic acid (PFNA), perfluorohexane sulfonic acid (PFHxS), perfluorobutane sulfonic acid (PFBS), and perfluorohexanoic acid (PFHxA), among others.

The molecular structures shown in FIG. 8 highlight the characteristic perfluorinated carbon backbone common to PFAS compounds, which contributes to their persistence in environmental and biological systems. The presence of functional groups such as carboxyl (—COOH) and sulfonic (—SO₃H) moieties differentiates various PFAS species, influencing their electrochemical behavior and interaction with sensing surfaces.

The electrochemical detection strategy described herein leverages these structural differences to achieve selective PFAS detection. As previously described in FIGS. 7A-7C, the UP-FASt sensor platform utilizes the interaction of PFAS functional groups with electrocatalytically active sites on the nickel-graphene composite electrode. The adsorption mechanisms, influenced by hydrophobic interactions, π-π stacking, and van der Waals forces, enable selective recognition of specific PFAS species.

The structures shown in FIG. 8 further underscore the regulatory importance of PFAS monitoring. The EPA's recent establishment of maximum contaminant levels (MCLs) for these compounds (4-10 ppt) has driven the need for ultra-trace detection technologies capable of field deployment. The electrochemical sensing methodology presented in this disclosure addresses this critical need by providing a rapid, selective, and cost-effective means of quantifying PFAS in environmental samples, as demonstrated in FIGS. 4A-4C.

By incorporating machine learning-based electrochemical signal processing, as shown in FIG. 7A-7C, and optimizing the sensor's working electrode composition, as shown in FIGS. 3A-3C, the present disclosure provides a robust analytical platform for detecting and quantifying the regulated PFAS compounds illustrated in FIG. 8.

### Test Kit System And Method

In accordance with an aspect of the present invention, a test kit system for the electrochemical detection of PFAS in an aqueous sample is provided.

### Kit Components

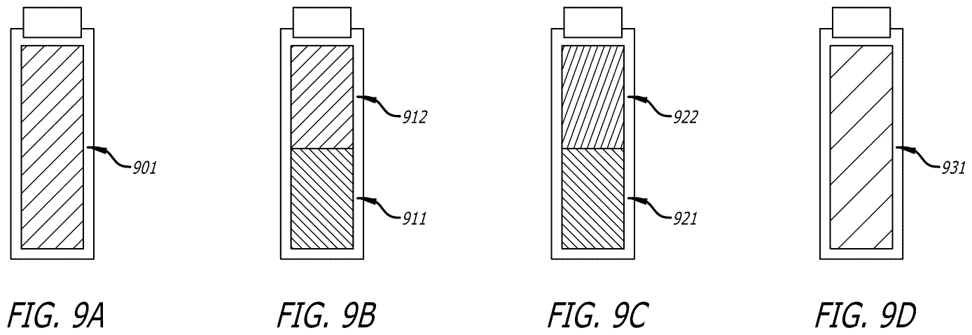

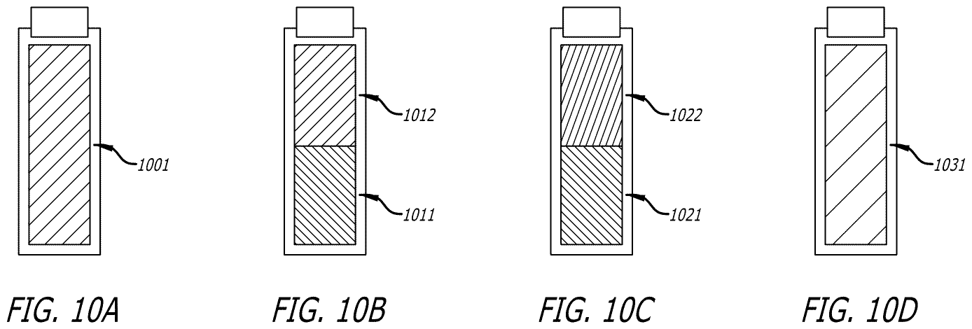

The test kit system can include four reagent solutions, designated herein as Kit A, Kit B, Kit C, and Kit D. Each kit component is provided in a sealed, pre-measured vial. The test kit components are illustrated in FIGS. 9A-9D (Kits A-D, respectively, for a 10 mL configuration) and FIGS. 10A-10D (Kits A-D, respectively, for a 5 mL configuration). Other configurations can involve test kit solutions of less or more volumes.

Kit A: Activation Solution. Kit A is an activation solution, such as a hydroxide electrolyte solution at a concentration of about 0.1 M KOH. Kit A shown in FIG. 9A contains an activation solution 901 of 10 mL of 0.1 M KOH; Kit A shown in FIG. 10A contains an activation solution 1001 of 5 mL of 0.1 M KOH. The activation solution is used for performing cyclic voltammetry (CV) to activate the NiOOH catalyst on the graphene-coated electrode prior to each chronoamperometric measurement. Kit A can be reused for multiple CV activation cycles within a single test session, as its composition does not change during the voltammetric activation. Kit A may be reused for rinsing/cleaning as the final step of a test as well.

Kit B: Blank Solution. Kit B is a blank solution, such as a pre-mixed blank solution containing the hydroxide electrolyte at a final measurement concentration. The blank solution can be prepared by mixing a hydroxide stock solution with PFAS-free deionized water in a predetermined volumetric ratio. Kit B shown in FIG. 9B contains a blank solution of 10 mL of 0.1 M KOH, which was prepared by pre-mixing 5.0 mL of 0.2 M KOH stock 911 with 5.0 mL of PFAS-free deionized water 912 to yield the 10.0 mL of 0.1 M KOH blank solution (1:1 ratio, 2×dilution). Kit B shown in FIG. 10B contains a blank solution of 5 mL of 0.1 M KOH, with 2.5 mL of 0.2 M KOH stock 1011 pre-mixed with 2.5 mL of PFAS-free deionized water 1012 to yield the 5.0 mL of 0.1 M KOH blank solution.

Kit C: Sample Solution Vessel. Kit C is a sample preparation vessel that contains a pre-measured volume, such as the hydroxide stock solution, with the remaining volume to be filled by the user with the aqueous sample to be tested. The hydroxide stock solution in Kit C is provided at a concentration and volume such that, upon addition of the aqueous sample, the final hydroxide concentration matches that of the blank solution of the kit (i.e., 10 mL for the kit of FIGS. 9A-9D and 5 mL for the kit of FIGS. 10A-10D). Kit C shown in FIG. 9C contains 5.0 mL of 0.2 M KOH stock 921, and the user can add 5.0 mL of the water sample 922 to be tested, yielding 10.0 mL of 0.1 M KOH containing the water sample at a 2× dilution factor. Kit C shown in FIG. 10C contains 2.5 mL of 0.2 M KOH stock 1021, and the user can add 2.5 mL of the water sample 1022, yielding 5.0 mL of 0.1 M KOH at the same 2× dilution.

Kit D: Cleaning Solution. Kit D is a cleaning solution, such as about 0.1 M KOH, for rinsing the electrode assembly (working, reference, and counter electrodes) after testing a sample. Kit D shown in FIG. 9D contains a cleaning solution 931 of 10 mL of about 0.1 M KOH; Kit A shown in FIG. 10D contains a cleaning solution 1031 of 5 mL of about 0.1 M KOH. The cleaning procedure ensures that residual PFAS from the sample does not carry over to subsequent measurements. In an embodiment, the cleaning is performed by immersing the electrode in the cleaning solution with electrode rotation at about 500 rpm for a duration of about 5 seconds or more. The specific cleaning duration and procedure can be optimized based on the PFAS concentration and sample matrix. In some embodiments, Kit D can contain methanol at concentrations of up to 1% to enable the cleaning of the electrodes before another test.

### Volume Ratios And Dilution Matching

An aspect of the present disclosure is the precise matching of dilution factors between the blank solution (Kit B, such as shown in FIGS. 9B and 10B) and the sample solution (Kit C, such as shown in FIGS. 9C and 10C). Because the electrochemical response of the NiOOH catalyst is sensitive to the hydroxide concentration (see EP 3 060 701 B1; FIGS. 4A-4C), even small differences in KOH concentration between the blank and sample solutions could produce current changes comparable to or exceeding the signal from PFAS at 4 ppt. The dilution-matching approach of the present disclosure ensures that the hydroxide concentration, ionic strength, and total volume are identical in both the blank and sample measurements.

The volumetric ratios are defined by the following relationship:

$$C\_stock \times V\_stock = C\_final \times V\_total \qquad ( 2 )$$

where C_stock is the concentration of the stock solution (such KOH stock solution), V_stock is the volume of stock in the vial, C_final is the target final concentration (such as the target final KOH concentration), and V_total is the total measurement volume.

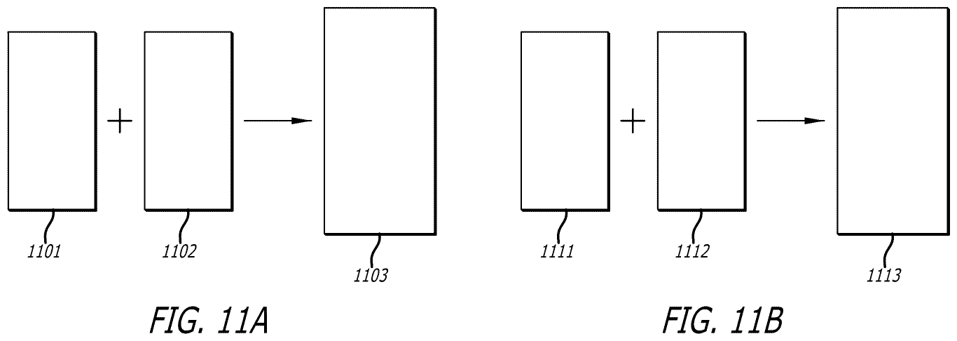

For example, as shown in FIG. 11A, the Kit B preparation of 10 mL of the blank sample 1103 is a mixture of 5 mL 0.2 M KOH 1101 and 5 ml DI water (PFAS-free) 1102 to form 10 mL 0.1 M KOH final. (C_final=(0.2 M×5 mL)/10 mL=0.1 M KOH). Further, the Kit C preparation of the 10 mL sample 1113 is a mixture of 5 mL 0.2 M KOH 1111 and 5 ml water sample 1112 to form 10 mL 0.1 M KOH final. (C_final=(0.2 M×5 mL)/10 mL=0.1 M KOH).

TABLE I shows some various volume configurations for the sample solution prepared in Kit C that can be used for PFAS sensor kits of the present disclosure.

TABLE I
Exemplary Volume Configurations For PFAS Sensor Test Kits

| | Total | Stock Vol | Water | Final | | |
|---|---|---|---|---|---|---|
| Configu- | Vol | (0.2 M | Sample | Conc | Dilution | Sample |
| ration | (Kit C) | KOH) | Vol | (KOH) | Ratio | Dilution |
| Standard | 10 mL | 5.0 mL | 5.0 mL | 0.1 M | 1:1 | 2x |
| Compact | 5 mL | 2.5 mL | 2.5 mL | 0.1 M | 1:1 | 2x |
| High-volume | 10 mL | 8.0 mL | 2.0 mL | 0.16 M | 4:1 | 5x |
| Low-volume | 10 mL | 2.0 mL | 8.0 mL | 0.04 M | 1:4 | 1.25x |
| Micro | 2 mL | 1.0 mL | 2.0 mL | 0.1 M | 1:1 | 2x |

For a blank solution of the same volume for the configuration (in Kit B), the final concentration can be the same by using the same amount of stock volume mixed with the DI water (PFAS-free) in the same volume as the water sample to be added. For instance, for Kit B, to be used with the “High-volume” configuration of Kit C shown in TABLE I, the total volume of the blank solution of 10 mL in Kit C will be a pre-mixture of (a) 8.0 mL of 2M KOH, and (b) 2.0 mL DI water (PFAS-free), which yields a final concentration of KOH in the blank solution of Kit B to be 0.16 M.

In certain embodiments, a 1:1 volumetric ratio (stock to water, such as 0.2 M KOH stock to water) is used, yielding a 2× dilution of the water sample. For a 0.2 M KOH stock, this 1:1 volumetric ratio can provide a balance between: (a) maintaining sufficient KOH concentration (0.1 M) for robust NiOOH catalyst activation, as supported by the electrochemical data in EP 3 060 701 B1; (b) minimizing the dilution of the PFAS analyte to preserve detection sensitivity; and (c) providing sufficient suppression of matrix effects from the water sample through the alkaline electrolyte.

Alternative volumetric ratios may be used depending on the application requirements. For example, volumetric ratios of 0.2M KOH to water can be used of (a) 4:1, which will yield 0.16 M KOH and 5× sample dilution, and (b) 1:4, which will yield 0.04 M KOH and 1.25× sample dilution.

### Kit Composition—Detailed Specifications

TABLE II provides detailed compositions for the representative 10 mL and 5 mL test kit configurations, in accordance with certain embodiments of the present disclosure.

TABLE II
Representative Detailed Kit Compositions For 10 mL And 5 mL
Configurations

| | Kit A | Kit B | Kit C Sample | Kit D |
|---|---|---|---|---|
| | Activation | Blank | Vessel | Cleaning |
| 10 mL | 10 mL of | 5.0 mL | 5.0 mL | 10 mL of |
| | 0.1M KOH | 0.2M | 0.2M | 0.1M KOH |
| | | KOH + | KOH + | (can contain |
| | | 5.0 mL | 5.0 mL | up to 1% v/v |
| | | DI H₂O | sample | methanol) |
| | | (pre-mixed) | (user adds) | |
| 5 mL | 5 mL of | 2.5 mL | 2.5 mL | 5 mL of |
| | 0.1M KOH | 0.2M | 0.2M | 0.1M KOH |
| | | KOH + | KOH + | (can contain |
| | | 2.5 mL | 2.5 mL | up to 1% v/v |
| | | DI H₂O | sample | methanol) |
| | | (pre-mixed) | (user adds) | |
| Final | 0.1M | 0.1M | 0.1M | 0.1M |
| [KOH] | | | | |
| Sample | N/A | 2× (DI | 2× (sample) | N/A |
| dilution | | water) | | |
| Reusable? | Yes (within | No | No | Yes (within |
| | session) | (single use) | (single use) | session) |

### Method of Use—Measurement Protocol

In accordance with certain embodiments of the present disclosure, methods for detecting PFAS in an aqueous sample using the test kit system is provided. The methods comprise a blank measurement phase and a sample measurement phase, wherein both phases follow an identical electrode preparation and measurement protocol to ensure that the only variable is the presence or absence of PFAS.

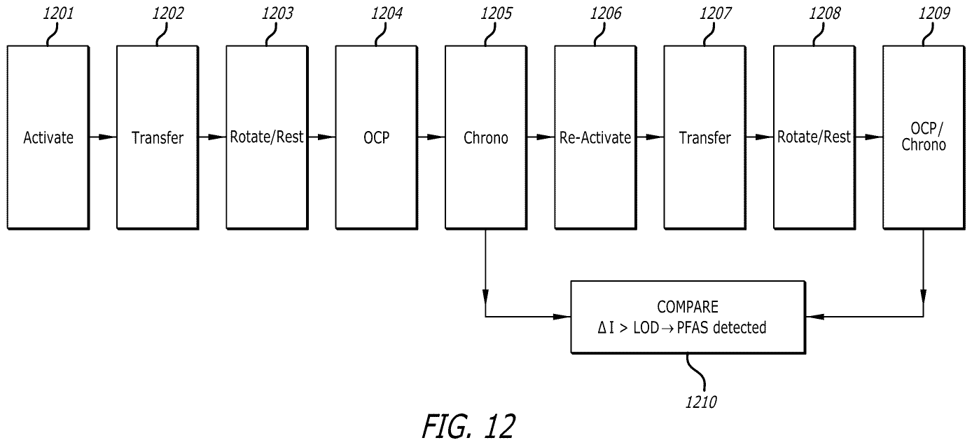

The measurement protocol for each phase (blank and sample) using a test kit of the present disclosure can include the steps shown in the PFAS test workflow depicted in FIG. 12.

In step 1201, Kit A is used for cyclic voltammetry activation, which can have a duration generally around 2 to 3 minutes. In such step, the user performs cyclic voltammograms. Generally, the number of cyclic voltammograms is generally around 5-10, and typically around 7, in the activation solution (Kit A) at a scan rate of about 20 mV/s over a potential window of about 0.2 to 0.58 V vs. Hg/HgO. The purpose of this step is to re-activate the NiOOH catalyst to a consistent surface state.

In step 1202, the electrode is transferred from Kit A to the measure solution that is Kit B for the blank solution. Exposure to air should be minimized. This transfer of the electrode typically takes about 5 seconds.

In step 1203, the start electrode is rotated (generally about 300 to 700 rpm, and typically around 500 rpm). During this step, no potential is applied. The purpose of this is to establish hydrodynamic steady state and to allow the electric double layer to equilibrate in the new solution. Generally, this rotation and rest step takes about 3 seconds.

In step 1204, the open circuit potential is measured. The user measures the open circuit potential (OCP) of the electrode. The OCP value will serve as a readiness criterion for the blank measurement. OCP should be below a threshold value (e.g., typically about 413 mV vs. Hg/HgO) and the OCP drift should be less than about 0.5 mV over the last 3 seconds. If either criterion is not met for the blank, additional activation cycles in Kit A can be performed. Generally, this step takes about 5 seconds.

In step 1205, chronoamperometry is performed. The user applies a potential step and records the current as a function of time. The analysis window can be from about t=5.0 to about t=7.0 seconds after the potential step, where the blank variability is minimized (standard deviation (SD)≈2 μA, limit of detection (LOD)≈6 μA). The primary metric is the current at t=~6.5 seconds; the secondary metric is the integrated charge from t=~5 to t=~7 seconds. Generally, this step takes about 7 seconds.

In step 1206, step 1201 is repeated for reactivation. As these were performed for the blank solution of Kit B), this reactivation can be performed in the blank solution (Kit B) as it has the same base electrolyte composition as Kit A. Alternatively, the activation solution of Kit A can be used. This step has a duration generally around 2 to 3 minutes.

For step 1207, step 1202 is repeated, except that the electrode is transferred to the measure solution that is Kit C for the sample solution. Again, this transfer of the electrode typically takes about 5 seconds.

For step 1208, step 1203 is repeated, i.e., the start electrode is rotated (generally about 300 to 700 rpm, and typically around 500 rpm), with no potential applied. Again, the purpose of this is to establish hydrodynamic steady state and to allow the electric double layer to equilibrate in the new solution. Generally, this rotation and rest step takes about 3 seconds.

For step 1209, steps 1204-1205 are repeated, i.e., the open circuit potential is measured, in which the OCP value will serve as a readiness criterion for the sample measurement, and chronoamperometry is performed, in which the user applies a potential step and records the current as a function of time. The primary metric is the current at t=~6.5 seconds; the secondary metric is the integrated charge from t=~5 to t=~7 seconds. Generally, this step takes about 7 seconds. This step 1209 should be repeated consistent with how steps 1204-1205 were performed to reduce outside factors from influencing the measurements recorded.

After the sample measurement in step 1209, all electrodes (working, reference, counter) are rinsed in the cleaning solution (Kit D) with rotation (again generally about 300 to 700 rpm, and typically around 500 rpm) for at least 5 seconds. The purpose of this is to remove residual PFAS to prevent carry-over.

In step 1210, the results recorded in steps 1205 and 1209 are compared. The greater the ΔI to the LOD, the more PFAS was detected.

Detection Method—Blank Vs. Sample Comparison

The presence or absence of PFAS in the aqueous sample is determined by comparing the chronoamperometric response of the blank measurement (step 1205) with the sample measurement (step 1209). Because both measurements follow identical electrode preparation, solution transfer, rest, OCP check, and chronoamperometry protocols, any systematic variability from electrode conditioning, hydrodynamic transients, or solution transfer is present in both measurements and cancels in the comparison.

For example, in an embodiment of the present disclosure, the comparison is performed at a time point of about 6.5 seconds after the potential step, where preliminary data indicate a blank standard deviation of about 2.04 μA (n=14 blanks). A current difference exceeding about 6 μA (corresponding to 3 times the standard deviation) between the blank and sample chronoamperometric curves is indicative of the presence of PFAS.

Further, in another embodiment of the present disclosure, the integrated charge from about t=5.0 to about t=7.0 seconds is compared, providing a charge-based metric with a coefficient of variation of about 1.37%.

In yet another embodiment of the present disclosure, a machine learning model trained on the full chronoamperometric curve shape (including multiple time points and the pre-measurement OCP value) was used for classification.

### Alternative Embodiments

In other embodiments, the hydroxide electrolyte comprises NaOH instead of KOH, at the same molar concentrations.

In other embodiments, the hydroxide electrolyte can include a mixture of KOH and a carbonate salt (e.g., K₂CO₃) to provide additional buffering capacity. The hydroxide concentration may range from about 0.04 M to about 0.5 M, with 0.1 M being preferred in certain embodiments as established by the electrochemical data supporting NiOOH catalyst performance at this concentration.

In other embodiments, the volumetric ratio of hydroxide stock to water sample is other than 1:1. For example, a 4:1 ratio (stock:sample) may be used when higher KOH concentration is desired for enhanced catalyst activation at the expense of greater sample dilution (5×). Conversely, a 1:4 ratio may be used when minimal sample dilution (1.25×) is desired to maximize analyte concentration, though at reduced KOH concentration (0.04 M). The selection of volumetric ratio represents a trade-off between electrocatalyst performance and analyte dilution that may be optimized for specific application requirements.

In other embodiments, the activation solution (Kit A) can include a higher concentration of KOH (e.g., 1 M KOH) for an initial activation phase comprising about 30 cyclic voltammograms, after which the activation solution is replaced with the 0.1 M KOH solution for the inter-measurement activation cycles of about 7 cyclic voltammograms. This two-stage activation protocol ensures thorough initial conditioning of the NiOOH catalyst while maintaining compatibility with the measurement electrolyte concentration.

In other embodiments, the test kit system Can further include a graphene-coated transition metal electrode, wherein the electrode is provided as a replaceable cartridge. The electrode may include nickel, cobalt, chromium, copper, manganese, zinc, or combinations thereof as the active catalyst material, with a graphene coating of about 1 to about 5 layers, as described in EP 3 060 701 B1. In certain embodiments, the active catalyst material can include nickel, and the graphene coating can be about 3 to 5 layers thick.

### Initial Electrode Activation

Before the first test in a session, the graphene-coated electrode can undergo an initial activation procedure. For instance, in certain embodiments, the initial activation includes about 30 cyclic voltammograms in 1 M KOH at a scan rate of about 20 mV/s, followed by rinsing in the activation solution (Kit A, 0.1 M KOH). Subsequently, two sacrificial measurement cycles (steps 1201-1205 using Kit B) can be performed and the resulting data discarded.

Data has shown that the first two measurement cycles can exhibit systematic deviations from subsequent measurements, with current values deviating up to 37.8% from the steady-state mean, attributable to incomplete catalyst conditioning. Discarding these first two cycles ensures that subsequent blank and sample measurements assist in these being performed with a fully conditioned electrode.

### Storage and Shelf Life Considerations

The test kit solutions can be stored in sealed, PFAS-free containers/vessels.

In embodiments, the containers can include high-density polyethylene (HDPE) or polypropylene vials with inert seals. The KOH stock solutions are stable indefinitely when sealed. The blank solution (Kit B) is prepared with PFAS-free deionized water and must be verified as PFAS-free to below 1 ppt prior to packaging. The sample solution vessel (Kit C) can contain only the KOH stock and is generally stable indefinitely when sealed; the sample is added by the user immediately prior to testing.

It should be appreciated that the container materials for the test kits should be selected to be free of PFAS contamination, as many conventional materials (including some PTFE-lined caps and fluoropolymer gaskets) may leach PFAS into the solutions. In certain embodiments, all containers, caps, and seals are verified as PFAS-free by independent analytical testing.

The foregoing description, for purposes of explanation, used specific nomenclature to provide a thorough understanding of the described embodiments. However, it should be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

While embodiments of the disclosure have been shown and described, modifications thereof can be made by one skilled in the art without departing from the spirit and teachings of the disclosure. The embodiments described and the examples provided herein are exemplary only, and are not intended to be limiting. Many variations and modifications of the disclosure disclosed herein are possible and are within the scope of the disclosure. The scope of protection is not limited by the description set out above, but is only limited by the claims which follow, that scope including all equivalents of the subject matter of the claims.

Embodiments can include a system, a method, and/or an apparatus.

Those skilled in the art will appreciate that the steps described herein may be carried out in a variety ways and that no particular ordering is required. It will be further understood from the foregoing description that modifications and changes may be made in various embodiments of the present disclosure without departing from its true spirit. The descriptions in this specification are for purposes of illustration only and are not to be construed in a limiting sense.

Consistent with the above disclosure, the examples of systems and methods enumerated in the following clauses are specifically contemplated and are intended as a non-limiting set of examples.

Clause 1. A system for detecting per- and polyfluoroalkyl substances (PFAS), including a working electrode, where the working electrode includes an electrode support, and a catalyst; a counter electrode, where the counter electrode includes a material selected from the group consisting of platinum, gold, iridium, nickel, titanium, rhodium, ruthenium, and combinations thereof; a reference electrode, where the reference electrode comprises a material selected from the group consisting of platinum, gold, silver, titanium, rhodium, ruthenium, mercury, mercury oxide, mercurous chloride, and combinations thereof; and a power source configured to apply a voltage between the working electrode and the reference electrode to facilitate electrochemical detection of PFAS.

Clause 2. The system of any foregoing clause, where the electrode support includes a conductive material.

Clause 3. The system of any foregoing clause, where the electrode support is selected from the group consisting of nickel, copper, titanium, zinc, stainless steel, corrosion-resistant nickel-based superalloys, graphite, and aluminum.

Clause 4. The system of any foregoing clause, where the electrode support is in the form of foil, plate, sheet, gauze, mesh, foam, or a combination thereof.

Clause 5. The system of any foregoing clause, where the catalyst includes a transition metal.

Clause 6. The system of any foregoing clause, where the transition metal is selected from the group consisting of nickel, iron, zinc, copper, cobalt, chromium, molybdenum, rhodium, ruthenium, palladium, iridium, and combinations thereof.

Clause 7. The system of any foregoing clause, where the catalyst includes a composite of graphene and at least one metal.

Clause 8. The system of any foregoing clause, where the electrode support and the catalyst are the same material.

Clause 9. The system of any foregoing clause, where the electrode support and catalyst include a material selected from the group consisting of nickel, iron, cobalt, chromium, copper, molybdenum, rhodium, ruthenium, palladium, iridium, and combinations thereof.

Clause 10. A system for real-time electrochemical detection of per- and polyfluoroalkyl substances (PFAS), including an electrochemical sensor having a working electrode, a counter electrode, and a reference electrode, where the working electrode comprises a transition metal or a graphene-metal composite; a controller configured to regulate an applied voltage and measure electrochemical response; a fluid handling system configured to transport a sample containing PFAS to the electrochemical sensor; and a data processing system configured to analyze the electrochemical response and determine PFAS concentration based on a calibration model.

Clause 11. The system of any foregoing clause, where the working electrode includes an electrode support and a catalyst.

Clause 12. The system of any foregoing clause, where the electrode support includes a conductive material.

Clause 13. The system of any foregoing clause, where the electrode support is selected from the group consisting of nickel, copper, titanium, zinc, stainless steel, Hastelloy, graphite, and aluminum.

Clause 14. The system of any foregoing clause, where the electrode support is in the form of foil, sheet, plate, gauze, mesh, foam, or a combination thereof.

Clause 15. The system of any foregoing clause, where the catalyst includes a transition metal.

Clause 16. The system of any foregoing clause, where the transition metal is selected from the group consisting of nickel, iron, zinc, copper, cobalt, chromium, molybdenum, rhodium, ruthenium, palladium, iridium, and combinations thereof.

Clause 17. The system of any foregoing clause, where the catalyst includes a composite of graphene and at least one metal.

Clause 18. The system of any foregoing clause, where the electrode support and catalyst are the same material.

Clause 19. The system of any foregoing clause, where the electrode support and catalyst include a material selected from the group consisting of nickel, iron, cobalt, chromium, copper, molybdenum, rhodium, ruthenium, palladium, iridium, and combinations thereof.

Clause 20. The system of any foregoing clause, where the counter electrode includes a material selected from the group consisting of platinum, gold, iridium, nickel, titanium, rhodium, ruthenium, and combinations thereof.

Clause 21. The system of any foregoing clause, where the reference electrode includes a material selected from the group consisting of platinum, gold, silver, titanium, rhodium, ruthenium, mercury, mercury oxide, mercurous chloride, and combinations thereof.

Clause 22. A method for detecting per- and polyfluoroalkyl substances (PFAS) in a sample, including inserting a sensor tip including a working electrode into an electrolyte solution or a sample containing PFAS; applying a voltage between the working electrode and a reference electrode; generating an electrocatalyst in situ at the working electrode through an electrochemical process; measuring a change in current; and correlating the measured change in current to the concentration of PFAS in the sample.

Clause 23. The method of any foregoing clause, where the applied voltage is within a range of −0.5 to 1 V versus the reference electrode.

Clause 24. The method of any foregoing clause, where the change in current is measured using differential pulse voltammetry (DPV).

Clause 25. The method of any foregoing clause, where the change in current is measured using chronoamperometry (CA).

Clause 26. The method of any foregoing clause, where the electrolyte solution includes at least one of potassium hydroxide (KOH), sodium hydroxide (NaOH), ammonium solutions, phosphate buffers, or a combination thereof.

Clause 27. The method of any foregoing clause, where the electrolyte solution includes at least one organic solvent selected from the group consisting of methanol, ethanol, isopropanol, and acetone.

Clause 28. The method of any foregoing clause, where the electrolyte solution has a pH between 9.5 and 14.

Clause 29. The method of any foregoing clause, where the sensor tip is inserted into the electrolyte solution.

Clause 30. The method of any foregoing clause, where the sensor tip rotates at a speed between 0 and 3000 rpm.

Clause 31. The method of any foregoing clause, where the temperature is controlled within a range of 10° C. to 40° C.

Clause 32. A test kit system for the electrochemical detection of per- and polyfluoroalkyl substances (PFAS) in an aqueous sample, wherein the test kit system includes an activation solution container including an activation solution including a hydroxide electrolyte at a first concentration suitable for cyclic voltammetric activation of a graphene-coated transition metal electrode; a blank solution container including a blank solution including the hydroxide electrolyte at a second concentration, wherein the second concentration is achieved by dilution of a first hydroxide stock solution with PFAS-free water in a first predetermined volumetric ratio, and the first hydroxide stock solution includes the hydroxide electrolyte; and a sample solution container including a second hydroxide stock solution, wherein the second hydroxide stock solution includes the hydroxide electrolyte, and the second hydroxide stock solution has a volume and concentration in the sample solution container such that, by addition of the aqueous sample at a second predetermined volumetric ratio to the second hydroxide stock solution in the sample solution container, a sample solution is formed in which the sample solution includes the hydroxide electrolyte at the second concentration, and dilution factor of the PFAS-free water in the blank solution is equal to dilution factor of the aqueous sample in the sample solution.

Clause 33. The test kit system of any foregoing clause, wherein the hydroxide electrolyte is selected from the group consisting of KOH, NaOH, and combinations thereof.

Clause 34. The test system of any foregoing clause, wherein the activation solution further includes an alkaline carbonate electrolyte, wherein the molar ratio of the hydroxide electrolyte and the alkaline carbonate electrolyte in the activation solution is at a predetermined hydroxide electrolyte/alkaline carbonate electrolyte ratio; the blank solution further includes the alkaline carbonate electrolyte, wherein the hydroxide electrolyte and the alkaline carbonate electrolyte in the blank solution is at the predetermined hydroxide electrolyte/alkaline carbonate electrolyte ratio; and the second hydroxide stock solution further includes the alkaline carbonate electrolyte, wherein the hydroxide electrolyte and the alkaline carbonate electrolyte in the second hydroxide stock solution is at the predetermined hydroxide electrolyte/alkaline carbonate electrolyte ratio.

Clause 35. The test kit system of any foregoing clause, wherein the first predetermined volumetric ratio and the second predetermined volumetric ratio are the same.

Clause 36. The test kit system of any foregoing clause, wherein the first predetermined volumetric ratio of hydroxide stock to water is between 1:4 and 4:1.

Clause 37. The test kit system of any foregoing clause, wherein the first predetermined volumetric ratio of hydroxide stock to water is about 1:1.

Clause 38. The test kit system of any foregoing clause, wherein the concentration of the first hydroxide stock solution is 0.2 M, and the concentration of the second hydroxide stock solution is 0.2 M.

Clause 39. The test kit system of any foregoing clause, wherein the second concentration is about 0.1 M.

Clause 40. The test kit system of any foregoing clause, wherein volume of the blank solution in the blank solution container is 5 mL or 10 mL; and volume of the sample solution after formation in the sample solution container is the same as the volume of the blank solution.

Clause 41. The test kit system of any foregoing clause further including a cleaning solution container including cleaning solution for removing residual PFAS from an electrode assembly after measurement of the sample solution.

Clause 42. The test kit system of any foregoing clause, wherein each of the activation solution container, the blank solution container, and the sample solution container is PFAS-free and includes high-density polyethylene and/or polypropylene.

Clause 43. A method for detecting PFAS in an aqueous sample using the test kit system of claim 32, wherein the method includes: activating a graphene-coated transition metal electrode by performing cyclic voltammetry utilizing the activation solution; transferring the electrode to the blank solution; allowing the electrode to rest at a first open circuit with rotation for at least 2 seconds; measuring a first open circuit potential for at least 5 seconds; performing a first chronoamperometry for at least 7 seconds, wherein first current data in response to the first chronoamperometry is recorded as a function of time; re-activating the graphene-coated transition metal electrode by performing cyclic voltammetry utilizing the activation solution or the blank solution; transferring the electrode to the sample solution; allowing the electrode to rest at a second open circuit with rotation for at least 2 seconds; measuring a second open circuit potential for at least 5 seconds; performing a second chronoamperometry for at least 7 seconds, wherein second current data in response to the second chronoamperometry is recorded as a function of time; and comparing the first current data to the second current data to detect PFAS in the aqueous sample.

Clause 44. The method of any foregoing clause, wherein the comparing the first current data to the second current data includes determining current difference between (a) the first current data at a time point of about 6.5 seconds after the first open circuit potential measurement step, and (b) the second current data at a time point of about 6.5 seconds after the second open circuit potential measurement step.

Clause 45. The method of any foregoing clause, wherein a determined current difference exceeding about 3 times blank standard deviation indicates presence of the PFAS.

Clause 46. The method of any foregoing clause, wherein the comparing the first current data to the second current data includes determining integrated charge from (a) about 5 seconds to about 7 seconds after the first open circuit potential measurement step and (b) about 5 seconds to about 7 seconds after the second open circuit potential measurement step.

Clause 47. The method of any foregoing clause, wherein the comparing the first current data to the second current data comprises an analysis in a first region, a second region, and a third region; the first region is an electrical double layer region; the second region is a transition region; and the third region is the Cottrell region.

Clause 48. The method of any foregoing clause, wherein the electrical double layer region is (i) at less than 1 second after the first open circuit potential measurement step for the first current data and (ii) at less than 1 second after the second open circuit potential measurement step for the second current data; the transition region is (i) between 1 second and 3 seconds after the first open circuit potential measurement step for the first current data and (ii) between 1 second and 3 seconds after the second open circuit potential measurement step for the second current data; and the Cottrell region is (i) over 3 seconds after the first open circuit potential measurement step for the first current data and (ii) over 3 seconds after the second open circuit potential measurement step for the second current data.

Clause 49. The method of any foregoing clause, wherein the comparing the first current data to the second current data yields a distinct fingerprint that is correlated to a type of the PFAS and a concentration of the PFAS.

Clause 50. The method of any foregoing clause further including a machine learning model trained to classify blank and sample chronoamperometric curves using features including one or more of the current at one or more time points, the integrated charge, the open circuit potential measured by the first the first open circuit potential measurement step, and the open circuit potential measured by the second open circuit potential measurement step.

Clause 51. The method of any foregoing clause further including, before the step of activating the graphene-coated transition metal electrode by performing cyclic voltammetry utilizing the activation solution, performing an initial activation step including at least 30 cyclic voltammograms in a hydroxide electrolyte solution having a concentration of at least 1 M, and followed by performing at least two sacrificial measurement cycles using the blank solution.

Clause 52. The method of any foregoing clause, wherein the electrode is allowed to rest at the first open circuit with rotation for at least 3 seconds; and the electrode is allowed to rest at the second open circuit with rotation for at least 3 seconds.

Clause 53. The method of any foregoing clause, wherein the step of detecting PFAS in the aqueous sample includes quantifying the amount of PFAS in the aqueous sample.

Clause 54. The method of any foregoing clause, wherein the graphene-coated transition metal electrode includes nickel, wherein the nickel is an active catalyst material, and a graphene coating including about 1 to about 5 layers of graphene.

Clause 55. The method of any foregoing clause, wherein the graphene coating including about 1 to about 5 layers of graphene was prepared by chemical vapor deposition.

### REFERENCES

Botte, G. Electrochemical Cell Containing a Graphene Coated Electrode. EP 3 060 701 B1, Jul. 18, 2018 (“EP 3 060 701 B1”).
Bresnahan, C. G., et al. “Exploration of Functionalizing Graphene and The Subsequent Impact on PFAS Adsorption Capabilities Via Molecular Dynamics, Chemosphere, 2023, 345, 140462.
Chan, M. G., et al. “Infrared reflection studies of the mechanism of oxidation at a copper—polyethylene interface,” Journal of Colloid and Interface Science, 1974, 47(3), 697-704.
Chow, C.-F., et al. “Combined Chemical Activation and Fenton Degradation to Convert Waste Polyethylene into High-Value Fine Chemicals,” Chemistry—A European Journal, 2016, 22(28), 9513-9518.
Clark, R. B., et al. “Electrochemical Sensing of Perfluorooctanesulfonate (PFOS) Using Ambient Oxygen in River Water,” ACS Sens., 2020, 5(11), 3591-3598.
Garg, S., et al. “Nano-Enabled Sensing of Per-/Poly-Fluoroalkyl Substances (PFAS) From Aqueous Systems—A Review,” J. Environ. Manage., 2022, 308, 114655.
Gecgel, O., et al. “Selective Electrochemical Detection of SARS-COV-2 Using Deep Learning,” Viruses, 2022, 14(9), 1930.
Hassan, M. H., et al. “Advances in Electrochemical Detection Methods for Measuring Contaminants of Emerging Concerns,” Electrochem. Sci. Adv., 2022, 2(6), e2100184.
Jafari, B., et al. “Nickel-Based Electrochemical Sensor with a Wide Detection Range for Measuring Hydroxyl Ions and pH Sensing,” J. Electroanal. Chem,” 2021, 895, 115547.
Jiang, X., et al. “Contribution of Nanobubbles for PFAS Adsorption on Graphene and OH- and NH2-Functionalized Graphene: Comparing Simulations with Experimental Results,” Environ. Sci. Technol., 2021, acs.est. 1c03022.
Karimian, N., et al. “Electrochemosensor for Trace Analysis of Perfluorooctanesulfonate in Water Based on a Molecularly Imprinted Poly(o-Phenylenediamine) Polymer,” ACS Sens. 2018, 3(7), 1291-1298.
Kazemi, R., et al. “Quantifying Interferent Effects on Molecularly Imprinted Polymer Sensors for Per- and Polyfluoroalkyl Substances (PFAS),” Anal. Chem., 2020, 92(15), 10597-10605.
Kotlarz, N., et al. “Measurement of Novel, Drinking Water-Associated PFAS in Blood from Adults and Children in Wilmington, North Carolina,” Environ. Health Perspect., 2020, 128(7), 077005.
Kotlarz, N., et al. “Reanalysis of PFO5DoA Levels in Blood from Wilmington, North Carolina, Residents, 2017-2018,” Environ. Health Perspect, 2024, 132(2), 027701.
Lu, F., et al. “Ammonia Generation via a Graphene-Coated Nickel Catalyst,” Coatings, 2017, 7(6), 72.
Lu, F., et al. “SARS-COV-2 Surveillance in Indoor Air Using Electrochemical Sensor for Continuous Monitoring and Real-Time Alerts,” Biosensors, 2022, 12(7), 523.
Menger, R., et al. “Sensors for Detecting Per- and Polyfluoroalkyl Substances (PFAS): A Critical Review of Development Challenges, Current Sensors, and Commercialization Obstacles,” Chem. Eng. J., 2021, 417, 129133.
Parthasarathy, V., et al. “Functionalization of Low Density Polyethylene with Ethyl Crotonate and Ethyl Salicylate in the Presence of Free Radical Initiator: FTIR-RI Kinetics Study,” Asian Journal of Chemistry, 2013, 25(7), 3755-3761.
Pelch, K. E., et al. “PFAS Health Effects Database: Protocol for a Systematic Evidence Map,” Environ. Int., 2019, 130, 104851.
Pervez, Md. N., et al. “Surface Modification of Graphene Oxide for Fast Removal of Per- and Polyfluoroalkyl Substances (PFAS) Mixtures from River Water,” ACS EST Water, 2024, 4(7), 2968-2980.
Ramanujam, A., et al. “Ultra-Fast Electrochemical Sensor for Point-of-Care COVID-19 Diagnosis Using Non-Invasive Saliva Sampling,” Processes, 2021, 9(7), 1236.
Ramanujam, A., et al. “Rapid Electrochemical Detection of Escherichia Coli Using Nickel Oxidation Reaction on a Rotating Disk Electrode,” Chem. Eng. J., 2021, 411, 128453.
Ringuette, A. E., et al. “Direct Functionalization of Polyethylene Surfaces with High-Density Polymer Brushes,” Journal of the American Chemical Society, 2024, 146 (30), 20563-20568.
Ritter, H., “Functionalized polymers: synthesis and properties,” Beilstein J. of Organic Chem., 2010, 6, 55.
Sack, S., et al. “Studies on the mechanism of the copper-catalyzed thermal oxidation of low-density polyethylene,” Polymer Degradation and Stability, 1984, 7(4), 193-203.
Sadia, M., et al. “Occurrence, Fate, and Related Health Risks of PFAS in Raw and Produced Drinking Water,” Environ. Sci. Technol., 2023, 57(8), 3062-3074.
Saha, D., et al. “Detection of Human Immunodeficiency Virus in Saliva Using a Nickel-Based Electrocatalyst,” ECS Sens. Plus, 2024, 3, 040602.
Sedaghat, S., “Development of a Nickel Oxide/Oxyhydroxide-Modified Printed Carbon Electrode as an All Solid-State Sensor for Potentiometric Phosphate Detection,” New J. Chem., 2019, 43(47), 18619-18628.
Shanbhag, M. M., “Detection of Perfluorooctanoic and Perfluorodecanoic Acids on a Graphene-Based Electrochemical Sensor Aided by Computational Simulations,” Langmuir, 2024, 40(7), 3831-3847.
Siddiqa, A. J., “Hydrophilic Low Density Polyethylene (LDPE) Films for Cell Adhesion and Proliferation,” Materials Science, 2015.
Teymoorian, T., “Tracking PFAS in Drinking Water: A Review of Analytical Methods and Worldwide Occurrence Trends in Tap Water and Bottled Water,” ACS EST Water, 2023, 3(2), 246-261.
Vedharathinam, V., “Understanding the Electro-Catalytic Oxidation Mechanism of Urea on Nickel Electrodes in Alkaline Medium,” Electrochimica Acta, 2012, 81, 292-300.
Zhan, J., “Non-Covalent Interactions of Graphene Surface: Mechanisms and Applications,” Chem, 2022, 8(4), 947-979.
Zhao, S., “Preparation of effective antibacterial composites of low-density polyethylene modified with quaternary ammonium functionalized zinc oxide nanoparticles,” J. of Polymer Research, 2024, 31(9), 262.

What is claimed is:

1. A test kit system for the electrochemical detection of per- and polyfluoroalkyl substances (PFAS) in an aqueous sample, wherein the test kit system comprises:
(a) an activation solution container comprising an activation solution comprising a hydroxide electrolyte at a first concentration suitable for cyclic voltammetric activation of a graphene-coated transition metal electrode;
(b) a blank solution container comprising a blank solution comprising the hydroxide electrolyte at a second concentration, wherein
(i) the second concentration is achieved by dilution of a first hydroxide stock solution with PFAS-free water in a first predetermined volumetric ratio, and
(ii) the first hydroxide stock solution comprises the hydroxide electrolyte; and
(c) a sample solution container comprising a second hydroxide stock solution, wherein
(i) the second hydroxide stock solution comprises the hydroxide electrolyte, and
(ii) the second hydroxide stock solution has a volume and concentration in the sample solution container such that, by addition of the aqueous sample at a second predetermined volumetric ratio to the second hydroxide stock solution in the sample solution container, a sample solution is formed in which
(i) the sample solution comprises the hydroxide electrolyte at the second concentration, and
(ii) dilution factor of the PFAS-free water in the blank solution is equal to dilution factor of the aqueous sample in the sample solution.

2. The test kit system of claim 1, wherein the hydroxide electrolyte is selected from the group consisting of KOH, NaOH, and combinations thereof.

3. The test system of claim 1, wherein
(a) the activation solution further comprises an alkaline carbonate electrolyte, wherein the molar ratio of the hydroxide electrolyte and the alkaline carbonate electrolyte in the activation solution is at a predetermined hydroxide electrolyte/alkaline carbonate electrolyte ratio;
(b) the blank solution further comprises the alkaline carbonate electrolyte, wherein the hydroxide electrolyte and the alkaline carbonate electrolyte in the blank solution is at the predetermined hydroxide electrolyte/alkaline carbonate electrolyte ratio; and
(c) the second hydroxide stock solution further comprises the alkaline carbonate electrolyte, wherein the hydroxide electrolyte and the alkaline carbonate electrolyte in the second hydroxide stock solution is at the predetermined hydroxide electrolyte/alkaline carbonate electrolyte ratio.

4. The test kit system of claim 1, wherein the first predetermined volumetric ratio and the second predetermined volumetric ratio are the same.

5. The test kit system of claim 4, wherein the first predetermined volumetric ratio of hydroxide stock to water is between 1:4 and 4:1.

6. The test kit system of claim 4, wherein the first predetermined volumetric ratio of hydroxide stock to water is about 1:1.

7. The test kit system of claim 6, wherein
(a) the concentration of the first hydroxide stock solution is 0.2 M, and
(b) the concentration of the second hydroxide stock solution is 0.2 M.

8. The test kit system of claim 1, wherein the second concentration is about 0.1 M.

9. The test kit system of claim 1, wherein
(a) the volume of the blank solution in the blank solution container is 5 mL or 10 mL; and
(b) the volume of the sample solution after formation in the sample solution container is the same as the volume of the blank solution.

10. The test kit system of claim 1 further comprising a cleaning solution container comprising cleaning solution for removing residual PFAS from an electrode assembly after measurement of the sample solution.

11. The test kit system of claim 1, wherein each of the activation solution container, the blank solution container, and the sample solution container is PFAS-free and comprises high-density polyethylene and/or polypropylene.

12. A method for detecting PFAS in an aqueous sample using the test kit system of claim 1, wherein the method comprises:
(a) activating a graphene-coated transition metal electrode by performing cyclic voltammetry utilizing the activation solution;
(b) transferring the electrode to the blank solution;
(c) allowing the electrode to rest at a first open circuit with rotation for at least 2 seconds;
(d) measuring a first open circuit potential for at least 5 seconds;
(e) performing a first chronoamperometry for at least 7 seconds, wherein first current data in response to the first chronoamperometry is recorded as a function of time;
(f) re-activating the graphene-coated transition metal electrode by performing cyclic voltammetry utilizing the activation solution or the blank solution;
(g) transferring the electrode to the sample solution;
(h) allowing the electrode to rest at a second open circuit with rotation for at least 2 seconds;
(i) measuring a second open circuit potential for at least 5 seconds;
(j) performing a second chronoamperometry for at least 7 seconds, wherein second current data in response to the second chronoamperometry is recorded as a function of time; and
(k) comparing the first current data to the second current data to detect PFAS in the aqueous sample.

13. The method of claim 12, wherein the comparing the first current data to the second current data comprises determining current difference between (a) the first current data at a time point of about 6.5 seconds after the first open circuit potential measurement step, and (b) the second current data at a time point of about 6.5 seconds after the second open circuit potential measurement step.

14. The method of claim 13, wherein a determined current difference exceeding about 3 times blank standard deviation indicates presence of the PFAS.

15. The method of claim 12, wherein the comparing the first current data to the second current data comprises determining integrated charge from (a) about 5 seconds to about 7 seconds after the first open circuit potential measurement step and (b) about 5 seconds to about 7 seconds after the second open circuit potential measurement step.

16. The method of claim 12, wherein
(a) the comparing the first current data to the second current data comprises an analysis in a first region, a second region, and a third region;
(b) the first region is an electrical double layer region;
(c) the second region is a transition region; and
(d) the third region is the Cottrell region.

17. The method of claim 16, wherein
(a) the electrical double layer region is (i) at less than 1 second after the first open circuit potential measurement step for the first current data and (ii) at less than 1 second after the second open circuit potential measurement step for the second current data,
(b) the transition region is (i) between 1 second and 3 seconds after the first open circuit potential measurement step for the first current data and (ii) between 1 second and 3 seconds after the second open circuit potential measurement step for the second current data, and
(c) the Cottrell region is (i) over 3 seconds after the first open circuit potential measurement step for the first current data and (ii) over 3 seconds after the second open circuit potential measurement step for the second current data.

18. The method of claim 17, wherein the comparing the first current data to the second current data yields a distinct fingerprint that is correlated to a type of the PFAS and a concentration of the PFAS.

19. The method of claim 12 further comprising a machine learning model trained to classify blank and sample chronoamperometric curves using features comprising one or more of the current at one or more time points, the integrated charge, the open circuit potential measured by the first the first open circuit potential measurement step, and the open circuit potential measured by the second open circuit potential measurement step.

20. The method of claim 12 further comprising, before the step of activating the graphene-coated transition metal electrode by performing cyclic voltammetry utilizing the activation solution,
(a) performing an initial activation step comprising at least 30 cyclic voltammograms in a hydroxide electrolyte solution having a concentration of at least 1 M; and
(b) followed by performing at least two sacrificial measurement cycles using the blank solution.

21. The method of claim 12, wherein
(a) the electrode is allowed to rest at the first open circuit with rotation for at least 3 seconds; and
(b) the electrode is allowed to rest at the second open circuit with rotation for at least 3 seconds.

22. The method of claim 12, wherein the step of detecting PFAS in the aqueous sample comprises quantifying the amount of PFAS in the aqueous sample.

23. The method of claim 12, wherein the graphene-coated transition metal electrode comprises:
(a) nickel, wherein the nickel is an active catalyst material; and
(b) a graphene coating comprising about 1 to about 5 layers of graphene.

24. The method of claim 23, wherein the graphene coating comprising about 1 to about 5 layers of graphene was prepared by chemical vapor deposition.
